(19) United States
(12) Patent Application Publication
(10) Pub. No.: US 2013/0261023 A1
(43) Pub. Date: Oct. 3, 2013

(54) SMALL MOLECULE PRINTING

(71) Applicants: David W. Barnes, Cambridge, MA (US); Angela N. Koehler, Cambridge, MA (US); James Elliot Bradner, Cambridge, MA (US); Ralph Mazitschek, Belmont, MA (US); Stuart L. Schreiber, Boston, MA (US)

(72) Inventors: David W. Barnes, Cambridge, MA (US); Angela N. Koehler, Cambridge, MA (US); James Elliot Bradner, Cambridge, MA (US); Ralph Mazitschek, Belmont, MA (US); Stuart L. Schreiber, Boston, MA (US)

(73) Assignees: Dana-Farber Cancer Institute, Inc., Boston, MA (US); President and Fellows of Harvard College, Cambridge, MA (US)

(21) Appl. No.: 13/648,667

(22) Filed: Oct. 10, 2012

Related U.S. Application Data
(63) Continuation of application No. 12/159,481, filed on Dec. 22, 2008, now abandoned.
(60) Provisional application No. 60/755,946, filed on Jan. 3, 2006.

(51) Int. Cl. G01N 33/68

(52) U.S. Cl. CPC G01N 33/6803
USPC 506/15

(57) ABSTRACT

The present invention provides compositions and methods to facilitate the identification of compounds that are capable of interacting with a biological macromolecule of interest. In one aspect, a composition is provided that comprises an array of one or more types of chemical compounds attached to a solid support using isocyanate or isothiocyanate chemistry, wherein the density of the array of compounds is at least 1000 spots per cm². In general, these inventive arrays are generated by: (1) providing a solid support, wherein said solid support is functionalized with an isocyanate or isothiocyanate moiety capable of interacting with a desired chemical compound to form a covalent attachment; (2) providing one or more solutions of one or more types of compounds to be attached to the solid support; (3) delivering said one or more types of compounds to the solid support; and (4) catalyzing the attachment of the compound to the solid support, whereby an array is formed and the array of compounds has a density of at least 1000 spots per cm². In another aspect, the present invention provides methods for utilizing these arrays to identify small molecule partners for biological macromolecules of interest.

### RELATED APPLICATIONS

The present application is a continuation of and claims priority under 35 U.S.C. §120 to U.S. patent application Ser. No. 12/159,481, filed Dec. 22, 2008, which is a national stage filing under 35 U.S.C. §371 of international PCT application, PCT/US2007/000003, filed Jan. 3, 2007, which claims priority under 35 U.S.C. §119(e) to U.S. provisional patent application, U.S. Ser. No. 60/755,946, filed Jan. 3, 2006, each of which is incorporated herein by reference.

### GOVERNMENT SUPPORT

This invention was made with Government support under the National Institutes of Health awards GM38637, AR049832, and 20XS139A. The Government has certain rights in this invention.

### BACKGROUND OF THE INVENTION

The ability to identify small molecule ligands for any protein or biomolecule of interest has far-reaching implications, both for the elucidation of protein function and for the development of novel pharmaceuticals. Natural products and products of diversity-oriented synthesis (DOS) and combinatorial chemistry constitute a rich pool of small molecules from which specific ligands to proteins or biomolecules of interest may be found (Schreiber, “Small molecules: the missing link in the central dogma” Nat. Chem. Biol. 2005; 1(2):64-66; incorporated herein by reference). In particular, with the introduction of split-pool strategies for synthesis (Furka et al., Int. J. Pept. Protein Res. 1991, 37, 487; Lam et al., Nature 1991, 354, 82; each of which is incorporated herein by reference) and the development of appropriate tagging technologies (Nestler et al., J. Org. Chem. 1994, 59, 4723; incorporated herein by reference), chemists are now able to prepare large collections of natural product-like compounds immobilized on polymeric synthesis beads (Tan et al., J. Am. Chem. Soc. 1998, 120, 8565; incorporated herein by reference). These libraries provide a rich source of molecules for the discovery of new ligands.

With such libraries of compounds in hand, the availability of efficient methods for screening these compounds becomes imperative. One method that has been used extensively is the on-bead binding assay (Lam et al., Chem. Rev. 1997, 97, 411; incorporated herein by reference). An appropriately tagged protein of interest is mixed with the library and beads displaying cognate ligands are subsequently identified by a chromagenic or fluorescence-based assay (Kapoor et al., J. Am. Chem. Soc. 1998, 120, 23; Morken et al., J. Am. Chem. Soc. 1998, 120, 30; St. Hilare et al., J. Am. Chem. Soc. 1998, 120, 13312; each of which is incorporated herein by reference). Despite the proven utility of this approach, it is limited by the small number of proteins that can be screened efficiently. In principle, the beads can be stripped of one protein and re-probed with another; however, this serial process is slow and limited to only a few iterations. In order to identify a specific small molecule ligand for every protein in a cell, tissue, or organism, high-throughput assays that enable each compound to be screened against many different proteins in a parallel fashion are required. Although Brown et al. (U.S. Pat. No. 5,807,522; incorporated herein by reference) have developed an apparatus and a method for forming high density arrays of biological macromolecules for large scale hybridization assays in numerous genetic applications, including genetic and physical mapping of genomes, monitoring of gene expression, DNA sequencing, genetic diagnosis, genotyping of organisms, and distribution of DNA reagents to researchers, the development of a high density array of natural product-like compounds for high-throughput screening has not been achieved.

In recent years, small-molecule microarrays (SMMs) have proven to useful in the discovery of previously unknown protein-ligand interactions, resulting in the identification of small-molecule modulators of protein function (Barnes-Seeman et al. Expanding the functional group compatibility of small-molecule microarrays: discovery of novel calmodulin ligands. Angew. Chem. Int. Ed. Engl. 2003; 42(21):2376-9; Fazio et al. Synthesis of sugar arrays in microtiter plate. J. Am. Chem. Soc. 2002; 124(48):14397-402; Hergenrother et al. Small molecule microarrays: covalent attachment and screening of alcohol-containing small molecules on glass slides. J. Am. Chem. Soc. 2000; 122:7849-50; Houseman et al. Carbohydrate arrays for the evaluation of protein binding and enzymatic modification. Chem. Biol. 2002; 9(4):443-54; Kanoh et al Immobilization of natural products on glass slides by using a photoaffinity reaction and the detection of protein-small-molecule interactions. Angew. Chem. Int. Ed. Engl. 2003; 42(45):5584-7; Kohn et al. Staudinger ligation: a new immobilization strategy for the preparation of small-molecule arrays. Angew. Chem. Int. Ed. Engl. 2003; 42(47):5830-4; MacBeath et al. Printing small molecules as microarrays and detecting protein-small molecule interactions en masse. J Am Chem Soc 1999; 121:7967-68; Uttamchandani et al. Microarrays of tagged combinatorial triazine libraries in the discovery of small-molecule ligands of human IgG. J. Comb. Chem. 2004; 6(6):862-8; Koehler et al. Discovery of an inhibitor of a transcription factor using small molecule microarrays and diversity-oriented synthesis. J. Am. Chem. Soc. 2003; 125(28):8420-1; Kuruvilla et al. Dissecting glucose signalling with diversity-oriented synthesis and small-molecule microarrays. Nature 2002; 416(6881):653-7; each of which is incorporated herein by reference). To make SMMs, stock solutions of compounds are robotically arrayed onto functionalized glass microscope slides that are then incubated with proteins or biomolecules of interest. Microarray features representing putative interactions between proteins and small molecules are typically visualized using fluorescently labeled antibodies and a standard fluorescence slide scanner.

Clearly, it would be desirable to develop methods for generating high density arrays that would enable the screening of compounds present in increasingly large and complex natural product-like combinatorial libraries in a high-throughput fashion to identify small molecule partners for biological macromolecules of interest.

### SUMMARY OF THE INVENTION

To date, several mild, selective coupling reactions have been used to covalently capture synthetic compounds onto glass surfaces and prepare small molecule microarrays. Exemplary reactions include a Michael addition (MacBeath et al. Printing small molecules as microarrays and detecting protein-small molecule interactions en masse. J. Am. Chem. Soc. 1999; 121:7967-68; U.S. Pat. No. 6,824,987, issued Nov. 30, 2004; U.S. patent application U.S. Ser. No. 10/998,867, filed Nov. 29, 2004, published as US 2005/0095639 on May 5, 2005; each of which is incorporated herein by reference), addition of a primary alcohol to a silyl chloride (Hergenrother et al. Small molecule microarrays: covalent attachment and screening of alcohol-containing small molecules on glass slides. J. Am. Chem. Soc. 2000; 122:7849-50; incorporated herein by reference), diazobenzylidene-mediated capture of phenols (Barnes-Seeman et al. Expanding the functional group compatibility of small-molecule microarrays: discovery of novel calmodulin ligands. Angew. Chem. Int. Ed. Engl. 2003; 42(21):2376-9; U.S. patent application U.S. Ser. No. 10/370,885, filed Feb. 20, 2003, published as US 2003/0215876 on Nov. 20, 2003; each of which is incorporated herein by reference), 1,3-dipolar cycloaddition (Fazio et al. Synthesis of sugar arrays in microtiter plate. J. Am. Chem. Soc. 2002; 124(48):14397-402; incorporated herein by reference), a Diels-Alder reaction (Houseman et al. Carbohydrate arrays for the evaluation of protein binding and enzymatic modification. Chem Biol 2002; 9(4):443-54; incorporated herein by reference), a Staudinger ligation of azides onto phosphane-modified slides (Kohn et al. Staudinger ligation: a new immobilization strategy for the preparation of small-molecule arrays. Angew Chem Int Ed Engl 2003; 42(47):5830-4; incorporated herein by reference), and capture of hydrazide-linked compounds onto epoxide-functionalized glass and vice-versa (Lee et al. Facile preparation of carbohydrate microarrays by site-specific, covalent immobilization of unmodified carbohydrates on hydrazide-coated glass slides. Org. Lett. 2005; 7(19):4269-72; Lee et al. Fabrication of Chemical Microarrays by Efficient Immobilization of Hydrazide-Linked Substances on Epoxide-Coated Glass Surfaces. Angew. Chem. Int. Ed. Engl. 2005; 44(19):2881-2884; each of which is incorporated herein by reference). Most of these surface-capture methods take advantage of a functional group, such as an alcohol or an azide, that is introduced as part of a solid-phase organic synthesis and biases the orientation of the small molecule on the surface (Kohn et al. Staudinger ligation: a new immobilization strategy for the preparation of small-molecule arrays. Angew. Chem. Int. Ed. Engl. 2003; 42(47):5830-4; Tallarico et al. An alkylsilyl-tethered, high-capacity solid support amenable to diversity-oriented synthesis for one-bead, one-stock solution chemical genetics. J. Comb. Chem. 2001; 3(3):312-8; each of which is incorporated herein by reference). Nonselective photoinduced cross-linking has also been used to immobilize a set of ten complex natural products onto glass slides (Kanoh et al. Immobilization of natural products on glass slides by using a photoaffinity reaction and the detection of protein-small-molecule interactions. Angew Chem Int Ed Engl 2003; 42(45):5584-7; incorporated herein by reference). Noncovalent approaches have also been employed, such as the hybridization of peptide-nucleic acid conjugates to oligonucleotide arrays (Winssinger et al. PNA-encoded protease substrate microarrays. Chem Biol 2004; 11(10):1351-60; Winssinger et al. Profiling protein function with small molecule microarrays. Proc Natl Acad Sci USA 2002; 99(17):11139-44; each of which is incorporated herein by reference).

Using selective approaches, we have immobilized over 50,000 products of diversity-oriented synthesis pathways via capture through primary alcohol on chlorinated slides or through capture of phenols on diazobenzylidene-functionalized slides (Barnes-Seeman et al. Expanding the functional group compatibility of small-molecule microarrays: discovery of novel calmodulin ligands. Angew Chem Int Ed Engl 2003; 42(21):2376-9; Hergenrother et al. Small molecule microarrays: covalent attachment and screening of alcohol-containing small molecules on glass slides. J. Am. Chem. Soc. 2000; 122:7849-50; Koehler et al. Discovery of an inhibitor of a transcription factor using small molecule microarrays and diversity-oriented synthesis. J. Am. Chem. Soc. 2003; 125(28):8420-21; each of which is incorporated herein by reference). Previous approaches have warranted the use of separate microarrays for compounds that contain either a primary alcohol or phenol. Additionally, we hoped to include compounds from natural sources, not necessarily bearing primary alcohols or phenols, alongside synthetic compounds in the microarrays. New capture strategies that would allow immobilization of several common functional groups that are present in both synthetic and natural compounds.

The present invention provides a system for the high-throughput screening of compounds for the identification of desirable properties or interactions. In a preferred embodiment, the present invention provides a system to facilitate the identification of chemical compounds that are capable of interacting with a biological macromolecule of interest. In one aspect, a composition is provided that comprises an array of more than one type of chemical compounds attached to a solid support using isocyanate chemistry as discussed herein. In certain embodiments, the density of the array of compounds is at least 500 spots per cm², at least 1000 spots per cm², at least 5000 spots per cm², or at least 10,000 spots per cm². In another aspect, a composition is provided that comprises a plurality of one or more types of non-oligomeric chemical compounds attached to a glass or polymer support using isocyanate chemistry, wherein the density of the array of compounds comprises at least 1000 spots per cm². In a particularly preferred embodiment, the chemical compounds are non-peptidic and non-oligomeric. In certain embodiments, the chemical compounds are small molecules. In certain embodiments, the chemical compounds are natural products. In certain embodiments, the chemical compounds are mixtures of chemical compounds (e.g., crude natural product extracts, mixtures of small molecules, etc.). The compounds are attached to the solid support through a covalent interaction via a reaction between a functional group on the chemical compounds being attached to the support and the isocyanate- or isothiocyanate-functionalized support. In a particular embodiment, the compounds are attached to a glass surface (e.g., glass slides) using the isocyanate or isothiocyanate chemistry discussed herein. In general, the inventive arrays are generated by: (1) providing a solid support, wherein said solid support is functionalized with an isocyanate or isothiocyanate moiety capable of interacting with a variety of functional groups to form a covalent attachment; (2) providing one or more solutions of one or more types of compounds to be attached to the solid support; (3) delivering said one or more types of compounds to the functionalized solid support; and (4) exposing the spotted support to a nucleophile (e.g., pyridine vapor), whereby an array of compounds covalently attached to the support is generated (FIG. 2). In certain embodiments, the array comprises a density of at least 1000 spots per cm². In other embodiments, the array comprises a density of at least 5000 spots per cm², and more preferably at least 10,000 spots per cm².

In one aspect, compounds are attached to a solid support using isocyanate chemistry as shown below:

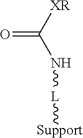

wherein

support is a solid support such as a glass surface, glass slide, polymeric support, plastic support, metal support, etc.;

L is a substituted or unsubstituted, branched or unbranched, cyclic or acyclic aliphatic or heteroaliphatic linker (e.g., polyethylene glycol spacer, polyethylene linker, —CH₂—, —CH₂CH₂—; —CH₂CH₂CH₂—, etc.);

n is an integer between 1 and 12, inclusive;

X is N, S, or O; and

R is the chemical compounds being attached to the solid support. The linkage is created by reacting a compound with an activated surface of formula:

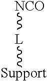

wherein L and Support are defined as above.

In certain particular embodiments, compounds are attached to a solid support through a linkage as shown below:

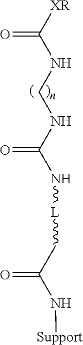

wherein

Support is a solid support such as a glass surface, glass slide, polymeric support, plastic support, metal support, etc.;

L is a substituted or unsubstituted, branched or unbranched, cyclic or acyclic aliphatic or heteroaliphatic linker (e.g., polyethylene glycol spacer, polyethylene linker, —CH₂—, —CH₂CH₂—; —CH₂CH₂CH₂—, etc.);

n is an integer between 1 and 12, inclusive;

X is N, S, or O; and

R is the chemical compounds being attached to the solid support. In certain embodiments, L is

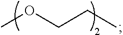

and n is 6.

In another aspect, compounds are attached to a solid support using isothiocyanate chemistry as shown below:

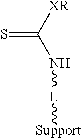

wherein

Support is a solid support such as a glass surface, glass slide, polymeric support, plastic support, metal support, etc.;

L is a substituted or unsubstituted, branched or unbranched, cyclic or acyclic aliphatic or heteroaliphatic linker (e.g., polyethylene glycol spacer, polyethylene linker, —CH₂—, —CH₂CH₂—; —CH₂CH₂CH₂—, etc.);

n is an integer between 1 and 12, inclusive;

X is N, S, or O; and

R is the chemical compounds being attached to the solid support. The linkage is created by reacting a compound with an activated surface of formula:

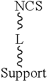

wherein L and Support are defined as above.

In certain embodiments, compounds are attached to a solid support through a linkage as shown below:

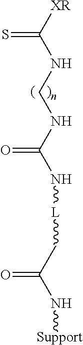

wherein

Support is a solid support such as a glass surface, glass slide, polymeric support, plastic support, metal support, etc.;

L is a substituted or unsubstituted, branched or unbranched, cyclic or acyclic aliphatic or heteroaliphatic linker (e.g., polyethylene glycol spacer, polyethylene linker, —CH₂—, —CH₂CH₂—; —CH₂CH₂CH₂—, etc.);

n is an integer between 1 and 12, inclusive;

X is N, S, or O; and

R is the chemical compounds being attached to the solid support. In certain embodiments, L is

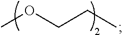

and n is 6.

In another aspect, the present invention provides an isocyanate functionalized solid support. In certain embodiments, the functional group on the solid support is of the formula:

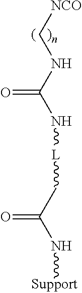

wherein

support is a solid support such as glass surface, glass slide, polymeric support, plastic support, metal support, etc.;

L is a substituted or unsubstituted, branched or unbranched, cyclic or acyclic aliphatic or heteroaliphatic linker (e.g., polyethylene glycol spacer, polyethylene linker, —CH₂—, —CH₂CH₂—; —CH₂CH₂CH₂—, etc.); and

n is an integer between 1 and 12, inclusive. In certain embodiments, L is

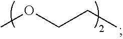

and n is 6.

In another aspect, the present invention provides an isothiocyanate functionalized solid support. In certain embodiments, the functional group on the solid support is of the formula:

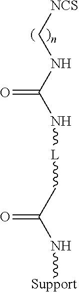

wherein

support is a solid support such as glass surface, glass slide, polymeric support, plastic support, metal support, etc.;

L is a substituted or unsubstituted, branched or unbranched, cyclic or acyclic aliphatic or heteroaliphatic linker (e.g., polyethylene glycol spacer, polyethylene linker, —CH₂—, —CH₂CH₂—; —CH₂CH₂CH₂—, etc.); and

n is an integer between 1 and 12, inclusive. In certain embodiments, L is

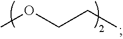

and n is 6.

In another aspect, the present invention provides methods for utilizing these arrays to identify small molecule partners for biological macromolecules (e.g., proteins, peptides, polynucleotides) of interest comprising: (1) providing an array of one or more types of compounds (e.g., more preferably, small molecules), wherein the array has a density comprising at least 1000 spots per cm²; (2) contacting the array with one or more types of biological macromolecules of interest; and (3) determining the interaction of specific small molecule-biological macromolecule partners. In preferred embodiments, the biological macromolecules of interest comprise a collection of one or more proteins or peptides. In particularly preferred embodiments, the biological macromolecules of interest comprise a collection of one or more recombinant proteins. In another preferred embodiment, the biological macromolecules of interest comprise a collection of macromolecules from a cell lysate (e.g., a bacterial cell lysate, yeast cell lysate, mammalian cell lysate, human cell lysate). In another preferred embodiment, the biological macromolecules of interest comprise a polynucleotide.

### DEFINITIONS

Unless indicated otherwise, the terms defined below have the following meanings:

“Aliphatic”: The term “aliphatic”, as used herein, includes both saturated and unsaturated, straight chain (i.e., unbranched), branched, acyclic, cyclic, or polycyclic aliphatic hydrocarbons, which are optionally substituted with one or more functional groups. As will be appreciated by one of ordinary skill in the art, “aliphatic” is intended herein to include, but is not limited to, alkyl, alkenyl, alkynyl, cycloalkyl, cycloalkenyl, and cycloalkynyl moieties. Thus, as used herein, the term “alkyl” includes straight, branched and cyclic alkyl groups. An analogous convention applies to other generic terms such as “alkenyl”, “alkynyl”, and the like. Furthermore, as used herein, the terms “alkyl”, “alkenyl”, “alkynyl”, and the like encompass both substituted and unsubstituted groups. In certain embodiments, as used herein, “lower alkyl” is used to indicate those alkyl groups (cyclic, acyclic, substituted, unsubstituted, branched or unbranched) having 1-6 carbon atoms.

In certain embodiments, the alkyl, alkenyl, and alkynyl groups employed in the invention contain 1-20 aliphatic carbon atoms. In certain other embodiments, the alkyl, alkenyl, and alkynyl groups employed in the invention contain 1-10 aliphatic carbon atoms. In yet other embodiments, the alkyl, alkenyl, and alkynyl groups employed in the invention contain 1-8 aliphatic carbon atoms. In still other embodiments, the alkyl, alkenyl, and alkynyl groups employed in the invention contain 1-6 aliphatic carbon atoms. In yet other embodiments, the alkyl, alkenyl, and alkynyl groups employed in the invention contain 1-4 carbon atoms. Illustrative aliphatic groups thus include, but are not limited to, for example, methyl, ethyl, n-propyl, isopropyl, cyclopropyl, —CH₂-cyclopropyl, vinyl, allyl, n-butyl, sec-butyl, isobutyl, tert-butyl, cyclobutyl, —CH₂-cyclobutyl, n-pentyl, sec-pentyl, isopentyl, tert-pentyl, cyclopentyl, —CH₂-cyclopentyl, n-hexyl, sec-hexyl, cyclohexyl, —CH₂-cyclohexyl moieties and the like, which again, may bear one or more substituents. Alkenyl groups include, but are not limited to, for example, ethenyl, propenyl, butenyl, 1-methyl-2-buten-1-yl, and the like. Representative alkynyl groups include, but are not limited to, ethynyl, 2-propynyl (propargyl), 1-propynyl, and the like.

“Antiligand”: As used herein, the term “antiligand” refers to the opposite member of a ligand/anti-ligand binding pair. The anti-ligand may be, for example, a protein or other macromolecule receptor in an effector/receptor binding pair.

“Compound”: The term “compound” or “chemical compound” as used herein can include organometallic compounds, organic compounds, metals, transitional metal complexes, and small molecules. In certain preferred embodiments, polynucleotides are excluded from the definition of compounds. In other preferred embodiments, polynucleotides and peptides are excluded from the definition of compounds. In a particularly preferred embodiment, the term compounds refers to small molecules (e.g., preferably, non-peptidic and non-oligomeric) and excludes peptides, polynucleotides, transition metal complexes, metals, and organometallic compounds.

“Cyclic”: The term “cyclic”, as used herein, refers to an aromatic or non-aromatic ring system. The ring system may be monocyclic or polycyclic (e.g., bicyclic, tricyclic, etc.). The rings may include only carbon atoms, or the rings may include multiple (e.g., one, two, three, four, five, etc.) heteroatoms such as N, O, P, or S. In a polycyclic ring system, the rings may be attached through aliphatic or heteroaliphatic linkages, the rings may be attached via a covalent carbon-carbon bond or carbon-heteroatom bond, the rings may be fused together, or the rings may be spiro-linked. The ring system may also be substituted.

“Heteroaliphatic”: The term “heteroaliphatic”, as used herein, refers to aliphatic moieties that contain one or more oxygen, sulfur, nitrogen, phosphorus, or silicon atoms, e.g., in place of carbon atoms. Heteroaliphatic moieties may be branched, unbranched, cyclic or acyclic and include saturated and unsaturated heterocycles such as morpholino, pyrrolidinyl, etc. In certain embodiments, heteroaliphatic moieties are substituted by independent replacement of one or more of the hydrogen atoms thereon with one or more moieties including, but not limited to aliphatic; heteroaliphatic; aryl; heteroaryl; arylalkyl; heteroarylalkyl; alkoxy; aryloxy; heteroalkoxy; heteroaryloxy; alkylthio; arylthio; heteroalkylthio; heteroarylthio; —F; —Cl; —Br; —I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; —NRₓ(CO)Rₓ, wherein each occurrence of Rₓ independently includes, but is not limited to, aliphatic, heteroaliphatic, aryl, heteroaryl, arylalkyl, or heteroarylalkyl, wherein any of the aliphatic, heteroaliphatic, arylalkyl, or heteroarylalkyl substituents described above and herein may be substituted or unsubstituted, branched or unbranched, cyclic or acyclic, and wherein any of the aryl or heteroaryl substituents described above and herein may be substituted or unsubstituted.

“Ligand”: As used herein, the term “ligand” refers to one member of a ligand/anti-ligand binding pair, and is referred to herein also as “small molecule”. The ligand or small molecule may be, for example, an effector molecule in an effector/receptor binding pair.

“Microarray”: As used herein, the term “microarray” is a regular array of regions, preferably spots of small molecule compounds, having a density of discrete regions of at least about 1000/cm².

“Natural Product-Like Compound”: As used herein, the term “natural product-like compound” refers to compounds that are similar to complex natural products which nature has selected through evolution. Typically, these compounds contain one or more stereocenters, a high density and diversity of functionality, and a diverse selection of atoms within one structure. In this context, diversity of functionality can be defined as varying the topology, charge, size, hydrophilicity, hydrophobicity, and reactivity to name a few, of the functional groups present in the compounds. The term, “high density of functionality”, as used herein, can preferably be used to define any molecule that contains preferably three or more latent or active diversifiable functional moieties. These structural characteristics may additionally render the inventive compounds functionally reminiscent of complex natural products, in that they may interact specifically with a particular biological receptor, and thus may also be functionally natural product-like.

“Peptide”: According to the present invention, a “peptide” comprises a string of at least three amino acids linked together by peptide bonds. Peptide may refer to an individual peptide or a collection of peptides. Inventive peptides preferably contain only natural amino acids, although non-natural amino acids (i.e., compounds that do not occur in nature but that can be incorporated into a polypeptide chain) and/or amino acid analogs as are known in the art may alternatively be employed. Also, one or more of the amino acids in an inventive peptide may be modified, for example, by the addition of a chemical entity such as a carbohydrate group, a phosphate group, a farnesyl group, an isofarnesyl group, a fatty acid group, a linker for conjugation, functionalization, or other modification, etc.

“Polynucleotide” or “oligonucleotide”: Polynucleotide or oligonucleotide refers to a polymer of nucleotides. The polymer may include natural nucleosides (i.e., adenosine, thymidine, guanosine, cytidine, uridine, deoxyadenosine, deoxythymidine, deoxyguanosine, and deoxycytidine), nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-methylcytidine, 7-deazaadenosine, 7-deazaguanosine, 8-oxoadenosine, 8-oxoguanosine, O(6)-methylguanine, and 2-thiocytidine), chemically modified bases, biologically modified bases (e.g., methylated bases), intercalated bases, modified sugars (e.g., 2′-fluororibose, ribose, 2′-deoxyribose, arabinose, and hexose), or modified phosphate groups (e.g., phosphorothioates and 5′-N-phosphoramidite linkages).

“Small Molecule”: As used herein, the term “small molecule” refers to a non-peptidic, non-oligomeric organic compound either synthesized in the laboratory or found in nature. Small molecules, as used herein, can refer to compounds that are “natural product-like”, however, the term “small molecule” is not limited to “natural product-like” compounds. Rather, a small molecule is typically characterized in that it contains several carbon-carbon bonds, and has a molecular weight of less than 1500, although this characterization is not intended to be limiting for the purposes of the present invention. Examples of “small molecules” that occur in nature include, but are not limited to, taxol, dynemicin, and rapamycin. Examples of “small molecules” that are synthesized in the laboratory include, but are not limited to, compounds described in Tan et al., (“Stereoselective Synthesis of over Two Million Compounds Having Structural Features Both Reminiscent of Natural Products and Compatible with Miniaturized Cell-Based Assays” J. Am. Chem. Soc. 1998, 120, 8565; incorporated herein by reference) and pending application Ser. No. 08/951,930, “Synthesis of Combinatorial Libraries of Compounds Reminiscent of Natural Products”, the entire contents of which are incorporated herein by reference. In certain other preferred embodiments, natural-product-like small molecules are utilized.

### DESCRIPTION OF THE DRAWING

FIG. 1 shows the schematic design of the diversity-SMM containing bioactive small molecules and products of diversity-oriented synthesis. Reactive functional groups are colored. Representative bioactive small molecules printed in the diversity array include 1a. nigericin 1b. bafilomycin A1 1c. doxorubicin 1d. genistein 1e. lactacystin 1f. uvaol 1g. D-erythro-sphingosine 1h. gibberellic acid 1i. ingenol 1j aloin. Representative scaffolds for DOS-small molecules printed in the diversity array include 2a. dihydropyrancarboxamides 2b. alkylidene-pyran-3-ones 2c. fused pyrrolidines 2d. serine-derived peptidomimetics 2e. shikimic acid-derived compounds 2f. 1,3-dioxanes 2g. spirooxindoles 2h. macrocyclic lactones 2i. ansa-seco steroid-derived compounds.

FIG. 2 depicts the vapor-catalyzed surface immobilization scheme. GAPS (γ-aminopropylsilane) slides (S1) are coated with a short Fmoc-protected polyethylene glycol spacer. After removal of the Fmoc group with piperidine, 1,6-diisocyanatohexane is coupled to the surface via urea bond formation to generate putative isocyanate-functionalized glass slides (S2). Slides printed with compound stock solutions are then placed in a dry environment and exposed to a pyridine vapor that catalyzes the covalent capture of small molecules onto the slide surface (S3).

FIG. 3 is a comparison of functional group reactivity with isocyanate-functionalized glass. (a) Parent structure of AP1497 derivatives 3a-3q. (b) AP1497 derivative array with FKBP12 ligands 3a-3q printed in serial two-fold dilutions (10 mM to 20 μM) onto isocyanate-derivatized slides. The slides were exposed to pyridine vapor to catalyze the attachment of printed compounds. Washed slides were probed with FKBP12-GST followed by a Cy5™-labeled anti-GST antibody. An image for a microarray scanned for fluorescence at 635 nm is shown. The functional groups presented for surface-capture are shown at the top of the array. (c) Total fluorescence intensity was computed within 300 μm spots centered on each microarray feature using GenePix Pro 6.0 microarray analysis software. The capture of small molecules is catalyzed in the presence of pyridine vapor and is tolerant of moisture in compound stock solutions. (d) Solutions of FKBP12 ligands 3a, 3d, 3e, 3r, and 3s (1 mM) in DMF were arrayed in triplicate onto surface S2 and the slides were incubated either under an atmosphere of N₂ (bottom) or in the presence of pyridine vapor under an atmosphere of N₂ (top). (e) Solutions of FKBP12 ligands 3a, 3d, 3h, and 3s (1 mM) in DMF (top row) or 9:1 DMF:ddH₂O (bottom row) were arrayed in triplicate onto isocyanate-derivatized slides.

FIG. 4 shows the detection of selected printed bioactives using antibodies. Fluorescence intensity relative to background signal for each printed bioactive is shown for binding profiles of (a) anti-corticosterone, (b) anti-digitoxin, and (c) anti-estradiol (rabbit) antibodies followed by Alexa Fluor® 647 goat-anti-rabbit, relative to (d) a Alexa Fluor® 647 goat-anti-rabbit IgG (A647 Rabbit) control. The signal-to-noise ratio at 635 nm (SNR635) is defined by (Mean Foreground−Mean Background)/(Standard Deviation of Background). Data represent mean values of duplicate spots on an individual array confirmed by two independent experiments. All compounds with SNR635 values greater than 3.0 are labeled.

FIG. 5 shows the screening of small-molecule microarrays with cellular lysates. (a) Schematic of the methodology. An epitope-tagged expression construct bearing a target protein of interest is introduced into a mammalian cell line by transient transfection. After 48 hrs replicate small-molecule microarrays are incubated serially with clarified lysate, primary anti-epitope antibody and finally a fluorophore-labeled secondary antibody. A gentle, brief wash is performed in PBS following each incubation. Fluorescence intensity is computed using GenePix Pro 6.0 microarray analysis software, and intensity relative to background signal (SNR635) for each printed small molecule is compared to replicate control arrays incubated with a cellular lysate from a mock-transfected, identical cell line. (b) Optimization of lysate screening methodology. Flag-FKBP12 over-expressed in HEK 293T cells and appropriate antibodies were selected for screening optimization experiments performed as depicted in (a) with FKBP12-ligand arrays patterned as identical triplicate subarrays with two-fold dilutions (10 mM to 20 μM) as described in FIG. 3b. Protocol conditions were serially optimized in a step-wise fashion. Data presented represent mean values (SNR635) of spots from triplicate subarrays. Data corresponding to FKBP12 derivatives 3a-3q (red) are compared to reference, blank DMSO spots (black) for experiments testing total protein concentration, the effects of blocking with bovine serum albumin (BSA), and polyethylene glycol (PEG) linker length.

FIG. 6 shows the detection of binding to ligands of varying affinity using cellular lysates. (a) Derivatives of AP1497 with varying affinities for FKBP12 (27, 28) were obtained and printed in quadruplicate with control compounds captopril and glutathione. (b) Arrays were incubated with clarified lysates of HEK-293T cells over-expressing Flag-FKBP12 and appropriate antibodies as depicted in FIG. 5a. A false-colored, representative image of an array scanned for fluorescence at 635 nm is shown. (c) Arrays were incubated with clarified lysates of HEK-293T cells over-expressing EGFP-FKBP12. A false-colored, demonstrative image of an array scanned for fluorescence at 488 nm is shown. (d) Arrays were incubated with clarified lysates of untransfected HEK-293T cells and probed with a polyclonal antibody against FKBP12. A false-colored, representative image of an array scanned for fluorescence at 635 nm is shown.

FIG. 7 shows the analysis of small-molecule microarrays screened with cellular lysates. (a) An array of 10,800 features was printed with a diverse set of known bioactives, natural products, AP1497 derivatives, and compounds prepared through diversity-oriented synthesis. DMSO solvent (n=158) was included for printing to determine hit threshold intensity. Five experiments with Flag-FKBP12 over-expressing cellular lysates were compared to five incubations with control, mock-transfected lysates. Each array was subsequently incubated with an anti-Flag monoclonal antibody and a secondary Cy5-labeled anti-mouse antibody. An FKBP12-probed array scanned for fluorescence at 532 nm (green) and 635 nm (red) is shown, as well as a highlighted region demonstrating binding to AP1497 derivatives. (b) Identification of FKBP12 binders. SNR635 profiles for five Flag-FKBP12 and five control arrays are shown. Each column is a sample on a discrete array (C, control; FK, Flag-FKBP12), and each row is a printed small molecule. The color scale indicates mean (0) and maximum (2.24) SNR635 for DMSO solvent spots. Printed molecules with SNR635 above the threshold established by printed solvent and satisfying a level of significance (p≦0.05) by Fisher's exact test are presented.

FIG. 8 shows the optimization of lysate screening methodology, complete data. Flag-FKBP12 over-expressed in HEK 293T cells and appropriate antibodies were selected for screening optimization experiments performed as depicted in FIG. 5a with FKBP12-ligand arrays patterned as identical triplicate subarrays with two-fold dilutions (10 mM to 20 μM) as described in FIG. 3b. Protocol conditions were serially optimized in a step-wise fashion. Data presented represent mean values (SNR635) of spots from triplicate subarrays. Data corresponding to FKBP12 derivatives 3a-3q (red) are compared to reference, blank DMF spots (blue) for experiments testing total protein concentration, the effects of blocking with bovine serum albumin (BSA), length of washing in PBS and polyethylene glycol (PEG) linker length. Also presented are a comparison of an alternative approach to printing via an ester linkage (MA), the utility of a labeled primary antibody for detection, and the utility of an alternate epitope for detection (hemagglutinin: HA).

FIG. 9 is (a) structure of 1276-M08, a spirooxindole DOS compound that was found to bind to FKBP12 from cell lysates. (b) sensorgram data for 1276-M08 binding to FKBP12-GST (left) and GST (right).

FIG. 10 is a flow diagram of a small molecule microarray (SMM) fabrication and screening process.

FIG. 11 shows a scheme for isocyanate-mediated immobilization of small molecules. Gamma-aminopropyl silane (GAPS) slides are coated with a short Fmoc-protected polyethylene glycol spacer. After deprotection using piperidine, 1,6-diisocyanatohexane is coupled to the surface via urea bond formation to provide the isocyanate-coated slides used during the microarraying process. Slides printed with small molecule stock solutions are exposed to pyridine vapor in order to catalyze the covalent attachment of molecules to the small molecule microarray (SMM) surface.

FIG. 12 shows a small molecule microarray probed with Flag-FKBP12 overexpressing cellular lysates. (a) Recognition of an analog of AP1497 printed through a primary amine. (b) Recognition of the natural product rapamycin, likely printed through a secondary alcohol. (c) Histogram of background-adjusted 635 nm fluorescence intensity data derived from solvent-only features on the SMM. (d) Histogram of background-adjusted 635 nm fluorescence intensity data derived from printed small molecule features on the SMM.

### DESCRIPTION OF CERTAIN PREFERRED EMBODIMENTS

The recent advances in the generation of complex chemical libraries of natural product-like compounds having as many as, or more than, one million members, has led to the subsequent need to facilitate the efficient screening of these compounds for biological activity. Towards this end, the present invention provides a system to enable the high-throughput screening of very large numbers of chemical compounds to identify those with desirable properties of interest. In certain embodiments, methods and compositions are provided to enable the high-throughput screening of very large numbers of chemical compounds to identify those compounds capable of interacting with biological macromolecules. In certain embodiments, the inventive screening system is used to identify a small molecule binding partner of a biological macromolecule of interest.

In one aspect, the present invention provides compositions comprising arrays of chemical compounds, attached to a solid support using isocyanate or isothiocyanate chemistry having a density of at least 1000 spots per cm², and methods for generating these arrays. In particular embodiments, the present invention provides arrays of small molecules, more preferably natural product-like compounds, that are generated from split-and-pool synthesis techniques, parallel synthesis techniques, and traditional one-at-a time synthesis techniques. In certain embodiments, the small molecules are mixtures of small molecules. In certain embodiments, the small molecules are natural products or extracts of natural products. The small molecules may be purified or partially purified. Additionally, existing collections of compounds may also be utilized in the present invention, to provide high density arrays that can be screened for compounds with desirable characteristics. In another aspect, the present invention provides methods for the identification of ligand (small molecule)-antiligand (biological macromolecule) binding pairs using the inventive chemical compound arrays based on isocyanate and isothiocyanate chemistry. It is particularly preferred that the antiligands be proteins, preferably recombinant proteins, and it is more particularly preferred that a library of recombinant proteins is utilized in the detection method.

In another embodiment, the antiligands comprise macromolecules from a cell lysate. Any cell may be used to prepare the lysate. For example, bacterial cells, human cells, yeast cells, mammalian cells, murine cells, nematode cells, fungal cells, plant cells, cancer cells, tumor cells, cells from laboratory cell lines, etc. In certain embodiments, a Streptomyces cell extract is utilized in the present invention. In certain embodiments, a mammalian cell extract is utilized in the present invention. In certain embodiments, a human cell extract is utilized in the present invention. The lysate may be prepared using any technique known in the art, e.g., sonication, homogenization, lysozyme treatment, French press, etc. The cell lysate may be used as is, or it may be partially purified before use in the inventive system. In certain embodiments, the cell lysate is clarified by centrifugation. In other embodiments, nucleic acids are removed before use of the lysate. In certain embodiments, the cell lysate is extracted with a solvent. In certain embodiments, a cell lysate is used in the inventive screening system.

### Small Molecule Printing

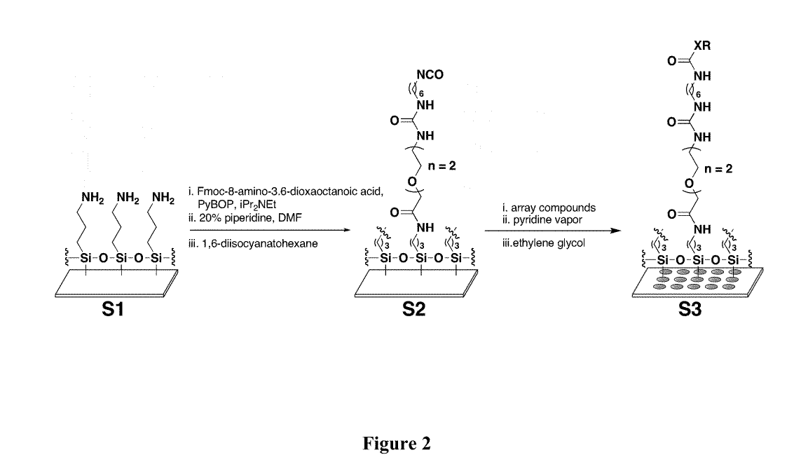

As discussed above, in one aspect, the present invention provides methods, referred to herein as “small molecule printing,” for the generation of high density arrays and the resulting compositions, wherein the small molecules are attached to a solid support using isocyanate chemistry (e.g., as illustrated in FIG. 2) or isothiocyanate chemistry.

According to the method of the present invention, a collection of chemical compounds, or one type of compound, is “printed” onto a support to generate high density arrays. In general, this method comprises (1) providing a solid support, wherein the solid support is functionalized with an isocyanate or isothiocyanate moiety capable of interacting with a desired chemical compound or collection of chemical compounds, to form an attachment(s); (2) providing one or more solutions of the same or different chemical compounds to be attached to the solid support; (3) delivering the one or more solutions of the same or different chemical compounds to the solid support; and (4) exposing the printed support to a nucleophile (e.g., pyridine vapor) that catalyzes the covalent capture of the small molecules onto the support, whereby an array of compounds is generated and the array has a density of at least 1000 spots per cm².

As one of ordinary skill in the art will realize, although any desired chemical compound capable of forming an attachment with the solid support may be utilized, it is particularly preferred that natural product-like compounds, preferably small molecules, particularly those generated from split-and-pool library or parallel syntheses are utilized. Examples of libraries of natural product-like compounds that can be utilized in the present invention include, but are not limited to shikimic acid-based libraries, as described in Tan et al. (“Stereoselective Synthesis of over Two Million Compounds Having Structural Features Both Reminiscent of Natural Products and Compatible with Miniaturized Cell-Based Assays”, J. Am. Chem. Soc., 1998, 120, 8565) and incorporated herein by reference. As will be appreciated by one of ordinary skill in the art, the use of split-and-pool libraries enables the more efficient generation and screening of compounds. However, small molecules synthesized by parallel synthesis methods and by traditional methods (one-at-a-time synthesis and modifications of these structures) can also be utilized in the compositions and methods of the present invention, as can naturally occurring compounds, or other collections of compounds, preferably non-oligomeric compounds, that are capable of attaching to a solid support without further synthetic modification. The compounds being attached to the microarrays may also be purchased from commercial sources such as Aldrich, Sigma, etc.

As will be realized by one of ordinary skill in the art, in split-and-pool techniques (see, for example, Furka et al., Abstr. 14th Int. Congr. Biochem., Prague, Czechoslovakia, 1988, 5, 47; Furka et al., Int. J. Pept. Protein Res. 1991, 37, 487; Sebestyen et al., Bioorg. Med. Chem. Lett. 1993, 3, 413; each of which is incorporated herein by reference), a mixture of related compounds can be made in the same reaction vessel, thus substantially reducing the number of containers required for the synthesis of very large libraries, such as those containing as many as or more than one million library members. As an example, a solid support bound scaffold can be divided into n vessels, where n represents the number of species of reagent A to be reacted with the support bound scaffold. After reaction, the contents from n vessels are combined and then split into m vessels, where m represents the number of species of reagent B to be reacted with the support bound scaffold. This procedure is repeated until the desired number of reagents are reacted with the scaffold structures to yield a desired library of compounds.

As mentioned above, the use of parallel synthesis methods are also applicable. Parallel synthesis techniques traditionally involve the separate assembly of products in their own reaction vessels. For example, a microtiter plate containing n rows and m columns of tiny wells which are capable of holding a small volume of solvent in which the reaction can occur, can be utilized. Thus, n variants of reactant type A can be reacted with m variants of reactant type B to obtain a library of n×m compounds.

Subsequently, once the desired compounds have been provided using an appropriate method, solutions of the desired compounds are prepared. In a preferred embodiment, compounds are synthesized on a solid support and the resulting synthesis beads are subsequently distributed into polypropylene microtiter plates at a density of one bead per well. In but one example, as discussed below in the Examples, the attached compounds are then released from their beads and dissolved in a small volume of suitable solvent. Due to the minute quantities of compound present on each bead, extreme miniaturization of the subsequent assay is required. Thus, in a particularly preferred embodiment, a high-precision transcription array robot (Schena et al., Science 1995, 270, 467; Shalon et al., Genome Research 1996, 6, 639; each of which is incorporated herein by reference) can be used to pick up a small volume of dissolved compound from each well and repetitively deliver approximately 0.1-10 nL of solution (e.g., approximately 0.01 mM to 20 mM) to defined locations on a series of isocyanate-functionalized glass microscope slides. The compounds may be provided as solutions in organic solvents such as DMF, DMSO, methanol, THF, etc. These isocyanate- or isothiocyanate-functionalized glass microscope slides are preferably prepared using custom slide-sized reaction vessels that enable the uniform application of solution to one face of the slide as shown and discussed in the Examples. This results in the formation of microscopic spots of compounds on the slides and in preferred embodiments these spots are 200-250 μm in diameter. It will be appreciated by one of ordinary skill in the art, however, that the current invention is not limited to the delivery of 1 nL volumes of solution and that alternative means of delivery can be used that are capable of delivering picoliter or smaller volumes. Hence, in addition to a high precision array robot (e.g., OmniGrid® 100 Microarrayer (Genomic Solutions)), other means for delivering the compounds can be used, including, but not limited to, ink jet printers, piezoelectric printers, and small volume pipetting robots.

As discussed, each compound contains a common functional group that mediates attachment to a support surface. It is preferred that the attachment formed is robust and therefore the formation of covalent ester, thioester, or amide attachments are particularly preferred. Isocyanate or isothiocyanate chemistry is employed to generate the high density arrays of chemical compounds. In addition to the robustness of the linkage, other considerations include the solid support to be utilized and the specific class of compounds to be attached to the support. Particularly preferred supports include, but are not limited to glass slides, polymer supports or other solid-material supports, and flexible membrane supports.

In another embodiment, as discussed in Example 1, the compounds are attached by nucleophilic addition of a functional group of the compounds being arrayed to an electrophile such as isocyanate or isothiocyanate. Functional groups found useful in adding to an isocyanate or isothiocyanate include primary alcohols, secondary alcohols, phenols, thiols, anilines, hydroxamic acid, aliphatic amines, primary amides, and sulfonamides. In certain embodiments, the nucleophilic addition reaction is catalyzed by a vapor such as pyridine. Other volatile nucleophilic reagents may also be used. In certain embodiments, the nucleophile includes an amine. In certain embodiments, a heteroaryl reagent is used. For example, the spotted slides may be dried and then exposed to pyridine vapor in a moisture-free environment (e.g., nitrogen atmosphere, argon atmosphere) in order to promote the attachment of the chemical compounds to the isocyanate- or isothiocyanate-derivatized solid support.

The slides are then optionally washed and dried. Slides prepared using the inventive method may be stored at −20° C. for months prior to screening. The slides may be prepared in a dessicator.

In one embodiment, compounds are attached to a solid support using isocyanate chemistry as shown in the formula:

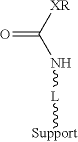

wherein

support is a solid support such as a glass surface, glass slide, polymeric support, plastic support, metal support, etc.;

L is a substituted or unsubstituted, branched or unbranched, cyclic or acyclic aliphatic or heteroaliphatic linker (e.g., polyethylene glycol spacer, polyethylene linker, —CH₂—, —CH₂CH₂—; —CH₂CH₂CH₂—, etc.);

X is N, S, or O; and

R is the chemical compound being attached to the solid support. The linkage is created by reacting a compound with an activated surface of formula:

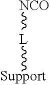

wherein L and Support are defined as above. The linker is 0 to 200 atoms in length, 0 to 100 atoms in length, 0 to 50 atoms in length, 2 to 50 atoms in length, 10 to 30 atoms in length, or 20 to 30 atoms in length. In certain embodiments, the linker is at least 2 atoms in length, at least 5 atoms in length, at least 10 atoms in length, or at least 20 atoms in length. In certain embodiments, the linker is acyclic. In other embodiments, the linker comprises cyclic moieties. For example, the linker may include an aryl, heteroaryl, carbocyclic, or heterocyclic moiety. In certain embodiments, the linker includes a phenyl ring. In certain embodiments, the linker is branched. In other embodiments, the linker is unbranched. In certain embodiments, the linker comprises heteroatoms including O, N, or S. In certain embodiments, the linker does not include heteroatoms. In certain embodiments, the linker includes carbonyl, ester, thioester, amide, carbonate, carbamoyl, or urea moieties. In certain embodiments, the linker includes halogen atoms.

In certain particular embodiments, compounds are attached to a solid support through a linkage as shown in the formula below:

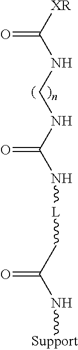

wherein

support is a solid support such as a glass surface, glass slide, polymeric support, plastic support, metal support, etc.;

L is a substituted or unsubstituted, branched or unbranched, cyclic or acyclic aliphatic or heteroaliphatic linker (e.g., polyethylene glycol spacer, polyethylene linker, —CH₂—, —CH₂CH₂—; —CH₂CH₂CH₂—, etc.);

n is an integer between 1 and 12, inclusive;

X is N, S, or O; and

R is the chemical compounds being attached to the solid support. In certain embodiments, L is

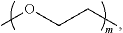

wherein m is an integer between 1 and 100, inclusive. In certain embodiments, m is an integer between 1 and 50, 1 and 25, 1 and 20, or 1 and 10, inclusive. In certain embodiments, m is 1, 2, 3, 4, 5, 6, 7, 8, 9, or 10. In certain embodiments, L is

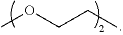

In certain embodiments, n is an integer between 1 and 100, inclusive. In certain embodiments, n is an integer between 1 and 50, 1 and 25, 1 and 20, or 1 and 10, inclusive. In certain embodiments, n is 1, 2, 3, 4, 5, 6, 7, 8, 9, or 10. In certain embodiments, n is 6. In certain embodiments, the linkage is of the formula:

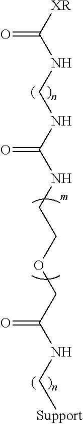

wherein

support is a solid support such as a glass surface, glass slide, polymeric support, plastic support, metal support, etc.;

each occurrence of n is an integer between 1 and 20, inclusive;

m is an integer between 1 and 20, inclusive;

X is N, S, or O; and

R is the chemical compounds being attached to the solid support. In certain embodiments, each occurrence of n and m is an integer between 1 and 10, inclusive. In certain embodiments, the support is a glass slide.

In certain particular embodiments, the linkage is of the formula:

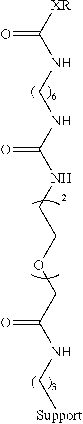

wherein

support is a solid support such as a glass surface, glass slide, polymeric support, plastic support, metal support, etc.;

X is N, S, or O; and

R is the chemical compounds being attached to the solid support.

The above compound arrays are prepared by attaching a compound to a support functionalized with an isocyanate moiety (i.e., —NCO). In certain embodiments, the isocyanate moiety is attached to the solid support via a linker. In certain embodiments, the linker is as shown above. In one aspect, the present invention provides an isocyanate-functionalized solid support (e.g., an isocyanate-functionalized glass slide).

In certain embodiments, the functional group on the solid support is of the formula:

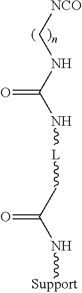

wherein

support is a solid support such as glass surface, glass slide, polymeric support, plastic support, metal support, etc.;

L is a substituted or unsubstituted, branched or unbranched, cyclic or acyclic aliphatic or heteroaliphatic linker (e.g., polyethylene glycol spacer, polyethylene linker, —CH₂—, —CH₂CH₂—; —CH₂CH₂CH₂—, etc.); and

n is an integer between 1 and 12, inclusive. In certain embodiments, L is

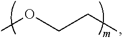

wherein m is an integer between 1 and 100, inclusive. In certain embodiments, m is an integer between 1 and 50, 1 and 25, 1 and 20, or 1 and 10, inclusive. In certain embodiments, m is 1, 2, 3, 4, 5, 6, 7, 8, 9, or 10. In certain embodiments, L is

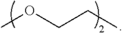

In certain embodiments, n is an integer between 1 and 100, inclusive. In certain embodiments, n is an integer between 1 and 50, 1 and 25, 1 and 20, or 1 and 10, inclusive. In certain embodiments, n is 1, 2, 3, 4, 5, 6, 7, 8, 9, or 10.

In certain embodiments, the functional group on the solid support is of the formula:

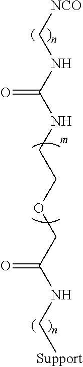

wherein

support is a solid support such as a glass surface, glass slide, polymeric support, plastic support, metal support, etc.;

each occurrence of n is independently an integer between 1 and 12, inclusive; and

m is an integer between 1 and 12, inclusive.

In certain particular embodiments, the linkage is of the formula:

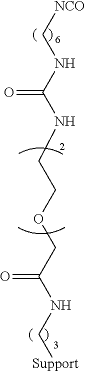

wherein

support is a solid support such as a glass surface, glass slide, polymeric support, plastic support, metal support, etc.

In another aspect, the present invention also provides a method of preparing functionalized supports comprising the steps of: functionalizing an amino group covalently linked to a support using 1,6-diisocyanatohexane. In certain embodiments, gamma-aminopropylsilane glass slides are coated with an amino-protected linker. The protecting groups is removed, and the free amino group is reacted with 1,6-diisocyanatohexane.

In another embodiment, compounds are attached to a solid support using isothiocyanate chemistry as shown in the formula:

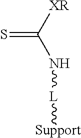

wherein

support is a solid support such as a glass surface, glass slide, polymeric support, plastic support, metal support, etc.;

L is a substituted or unsubstituted, branched or unbranched, cyclic or acyclic aliphatic or heteroaliphatic linker (e.g., polyethylene glycol spacer, polyethylene linker, —CH₂—, —CH₂CH₂—; —CH₂CH₂CH₂—, etc.);

n is an integer between 1 and 12, inclusive;

X is N, S, or O; and

R is the chemical compound being attached to the solid support. In certain embodiments, L is a cyclic aliphatic or heteroaliphatic linker. In certain embodiments, L is an aryl linker. In certain particular embodiments, L is a phenyl moiety, which may be substituted or unsubstituted. In certain embodiments, L is a para-substituted phenyl moiety. The linkage is created by reacting a compound with an activated surface of formula:

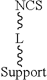

wherein L and Support are defined as above. The linker is 0 to 200 atoms in length, 0 to 100 atoms in length, 0 to 50 atoms in length, 2 to 50 atoms in length, 10 to 30 atoms in length, or 20 to 30 atoms in length. In certain embodiments, the linker is at least 2 atoms in length, at least 5 atoms in length, at least 10 atoms in length, or at least 20 atoms in length. In certain embodiments, the linker is a cyclic. In other embodiments, the linker comprises cyclic moieties. In certain embodiments, the linker is branched. In other embodiments, the linker is unbranched. In certain embodiments, the linker comprises heteroatoms including O, N, or S. In certain embodiments, the linker does not include heteroatoms. In certain embodiments, the linker includes carbonyl, ester, thioester, amide, carbonate, carbamoyl, or urea moieties. In certain embodiments, the linker includes halogen atoms.

In certain embodiments, compounds are attached to a solid support through a linkage as shown in the formula below:

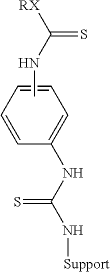

wherein

X is O, S, or N; and

R is an attached compound.

In certain particular embodiments, compounds are attached to a solid support through a linkage as shown in the formula below:

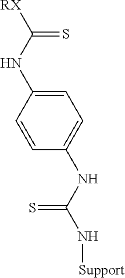

wherein

X is O, S, or N; and

R is an attached compound.

In certain particular embodiments, compounds are attached to a solid support through a linkage as shown in the formula below:

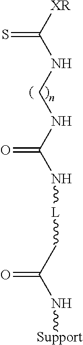

wherein

support is a solid support such as a glass surface, glass slide, polymeric support, plastic support, metal support, etc.;

L is a substituted or unsubstituted, branched or unbranched, cyclic or acyclic aliphatic or heteroaliphatic linker (e.g., polyethylene glycol spacer, polyethylene linker, —CH₂—, —CH₂CH₂—; —CH₂CH₂CH₂—, etc.);

n is an integer between 1 and 12, inclusive;

X is N, S, or O; and

R is the chemical compounds being attached to the solid support. In certain embodiments, L is

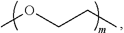

wherein m is an integer between 1 and 100, inclusive. In certain embodiments, m is an integer between 1 and 50, 1 and 25, 1 and 20, or 1 and 10, inclusive. In certain embodiments, m is 1, 2, 3, 4, 5, 6, 7, 8, 9, or 10. In certain embodiments, L is

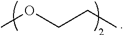

In certain embodiments, n is an integer between 1 and 100, inclusive. In certain embodiments, n is an integer between 1 and 50, 1 and 25, 1 and 20, or 1 and 10, inclusive. In certain embodiments, n is 1, 2, 3, 4, 5, 6, 7, 8, 9, or 10. In certain embodiments, n is 6. In certain embodiments, the linkage is of the formula:

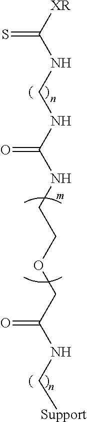

wherein

support is a solid support such as a glass surface, glass slide, polymeric support, plastic support, metal support, etc.;

each occurrence of n is an integer between 1 and 20, inclusive;

m is an integer between 1 and 20, inclusive;

X is N, S, or O; and

R is the chemical compounds being attached to the solid support. In certain embodiments, each occurrence of n and m is an integer between 1 and 10, inclusive. In certain embodiments, the support is a glass slide.

In certain particular embodiments, the linkage is of the formula:

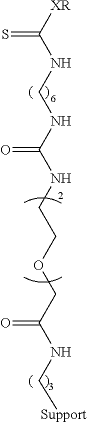

wherein

support is a solid support such as a glass surface, glass slide, polymeric support, plastic support, metal support, etc.;

X is N, S, or O; and

R is the chemical compounds being attached to the solid support.

The above compound arrays are prepared by attaching a compound to a support functionalized with an isothiocyanate moiety (i.e., —NCS). In certain embodiments, the isothiocyanate moiety is attached to the solid support via a linker. In certain embodiments, the linker is as shown above. In one aspect, the present invention provides an isothiocyanate-functionalized solid support (e.g., an isothiocyanate-functionalized glass slide).

In certain embodiments, the functional group on the solid support is of the formula:

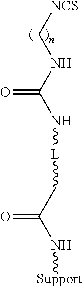

wherein

support is a solid support such as glass surface, glass slide, polymeric support, plastic support, metal support, etc.;

L is a substituted or unsubstituted, branched or unbranched, cyclic or acyclic aliphatic or heteroaliphatic linker (e.g., polyethylene glycol spacer, polyethylene linker, —CH₂—, —CH₂CH₂—; —CH₂CH₂CH₂—, etc.); and

n is an integer between 1 and 12, inclusive. In certain embodiments, L is

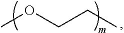

wherein m is an integer between 1 and 100, inclusive. In certain embodiments, m is an integer between 1 and 50, 1 and 25, 1 and 20, or 1 and 10, inclusive. In certain embodiments, m is 1, 2, 3, 4, 5, 6, 7, 8, 9, or 10. In certain embodiments, L is

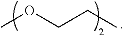

In certain embodiments, n is an integer between 1 and 100, inclusive. In certain embodiments, n is an integer between 1 and 50, 1 and 25, 1 and 20, or 1 and 10, inclusive. In certain embodiments, n is 1, 2, 3, 4, 5, 6, 7, 8, 9, or 10.

In certain embodiments, the functional group on the solid support is of the formula:

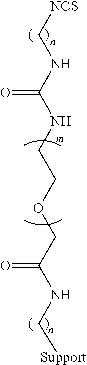

wherein

support is a solid support such as a glass surface, glass slide, polymeric support, plastic support, metal support, etc.;

each occurrence of n is independently an integer between 1 and 12, inclusive; and

m is an integer between 1 and 12, inclusive.

In certain particular embodiments, the linkage is of the formula:

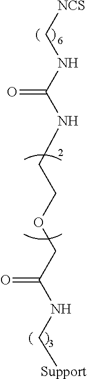

wherein

support is a solid support such as a glass surface, glass slide, polymeric support, plastic support, metal support, etc.

### Methods for Detecting Biological Activity

It will be appreciated by one of ordinary skill in the art that the generation of arrays of compounds having extremely high spatial densities facilitates the detection of binding and/or activation events occurring between compounds in a specific chemical library and biological macromolecules. Thus, the present invention provides, in yet another aspect, a method for identifying small molecule partners for biological macromolecules of interest. The partners may be compounds that bind to particular macromolecules of interest and are capable of activating or inhibiting the biological macromolecules of interest. In general, this method involves (1) providing an array of one or more types of compounds, as described above, wherein the array of small molecules has a density of at least 1000 spots per cm²; (2) contacting the array with one or more types of biological macromolecules of interest; and (3) determining the interaction of specific small molecule-biological macromolecule partners.

It will also be appreciated that the arrays of the present invention may be utilized in a variety of ways to enable detection of interactions between small molecules and biological macromolecules. In one particularly preferred embodiment, an array of different types of chemical compounds attached to the surface is utilized and is contacted by one or a few types of biological macromolecules to determine which compounds are capable of interacting with the specific biological macromolecule(s). As one of ordinary skill in the art will realize, if more than one type of compound is utilized, it is desirable to utilize a method for encoding each of the specific compounds so that a compound having a specific interaction can be identified. Specific encoding techniques have been recently reviewed and these techniques, as well as other equivalent or improved techniques, can be utilized in the present invention (see, Czarnik, A. W. Current Opinion in Chemical Biology 1997, 1, 60; incorporated herein by reference). Alternatively the arrays of the present invention may comprise one type of chemical compound and a library of biological macromolecules may be contacted with this array to determine the ability of this one type of chemical compound to interact with a variety of biological macromolecules. As will be appreciated by one of ordinary skill in the art, this embodiment requires the ability to separate regions of the support, utilizing paraffin or other suitable materials, so that the assays are localized.

As one of ordinary skill in the art will realize, the biological macromolecule of interest may comprise any biomolecule. In preferred embodiments, the biological macromolecule of interest comprises a protein, and more preferably the array is contacted with a library of recombinant proteins of interest. In yet another preferred embodiment, the biological molecules of interest are provided in the form of cell lysates such as those of tumor-associated cells. As will be appreciated by one of ordinary skill in the art, these proteins may comprise purified proteins, pools of purified proteins, and complex mixtures such as cell lysates, and fractions thereof, to name a few. Examples of particularly preferred biological macromolecules to study include, but are not limited to those involved in signal transduction, dimerization, gene regulation, cell cycle and cell cycle checkpoints, and DNA damage checkpoints. Furthermore, the ability to construct libraries of expressed proteins from any organism or tissue of interest will lead to large arrays of recombinant proteins. The compounds of interest may be capable of either inactivating or activating the function of the particular biomolecule of interest.

Each of the biological macromolecules may be modified to enable the facile detection of these macromolecules and the immobilized compounds. This may be achieved by tagging the macromolecules with epitopes that are subsequently recognized, either directly or indirectly, by a different receptor (e.g., an antibody) that has been labeled for subsequent detection (e.g., with radioactive atoms, fluorescent molecules, colored compounds, or enzymes that enable color formation, or light production, to name a few). Alternatively, the macromolecules themselves may be labeled directly using any one or other of these methods or not labeled at all if an appropriate detection method is used to detect the bound protein (e.g., mass spectrometry, surface plasmon resonance, and optical spectroscopy, to name a few).

In a particularly preferred embodiment, the inventive arrays are utilized to identify compounds for chemical genetic research. In classical genetics, either inactivating (e.g., deletion or “knock-out”) or activating (e.g., oncogenic) mutations in DNA sequences are used to study the function of the proteins that are encoded by these genes. Chemical genetics instead involves the use of small molecules that alter the function of proteins to which they bind, thus either inactivating or activating protein function. This, of course, is the basis of action of most currently approved small molecule drugs. The present invention involves the development of “chip-like” technology to enable the rapid detection of interactions between small molecules and specific proteins of interest. The examples presented below demonstrate how the methods and compositions of the present invention can be used to identify new small molecule ligands for use in chemical genetic research. One of ordinary skill in the art will realize that the inventive compositions and methods can be utilized for other purposes that require a high density chemical compound format.

As will also be appreciated by one of ordinary skill in the art, arrays of chemical compounds may also be useful in detecting interactions between the compounds and alternate classes of molecules other than biological macromolecules. For example, the arrays of the present invention may also be useful in the fields of catalysis and materials research to name a few.

These and other aspects of the present invention will be further appreciated upon consideration of the following Examples, which are intended to illustrate certain particular embodiments of the invention but are not intended to limit its scope, as defined by the claims.

### EXAMPLES

### Example 1

### A Robust Small-Molecule Microarray Platform for Screening Cell Lysates

Here we describe the preparation and use of isocyanate-functionalized glass slides to capture DOS compounds coming from various solid-phase organic synthesis routes and bioactive compounds, including natural products. Isocyanates react with a number of nucleophilic functional groups without leaving an acidic byproduct (Vandenabeele-Trambouze et al. Reactivity of organic isocyanates with nucleophilic compounds: amines, alcohols, thiols, oximes, and phenols in dilute organic solutions. Advanced Environmental Research 2001; 6:45-55; incorporated herein by reference) and an isocyanate surface thereby increases the diversity of small molecules, from natural or synthetic sources, that can be immobilized onto a single small molecule microarray (SMM). Isocyanate glass substrates have been prepared and used to immobilize oligonucleotides in a microarray format (Ameringer et al. Ultrathin functional star PEG coatings for DNA microarrays. Biomacromolecules 2005; 6(4):1819-23; Chun et al. Diisocyanates as novel molecular binders for monolayer assembly of zeolite crystals on glass. Chem Commun (Camb) 2002(17):1846-7; Guo et al. Direct fluorescence analysis of genetic polymorphisms by hybridization with oligonucleotide arrays on glass supports. Nucleic Acids Res. 1994; 22(24):5456-65; Sompuram et al. A water-stable protected isocyanate glass array substrate. Anal. Biochem. 2004; 326(1):55-68; each of which is incorporated herein by reference).

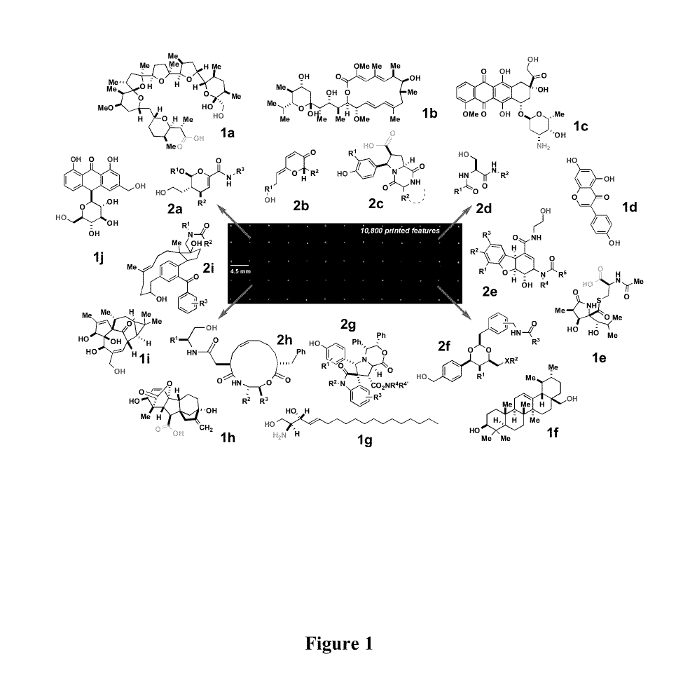

We have previously reported the use of SMMs to discover ligands for calmodulin (calmoduphilins) (Barnes-Seeman et al. Expanding the functional group compatibility of small-molecule microarrays: discovery of novel calmodulin ligands. Angew Chem Int Ed Engl 2003; 42(21):2376-9; incorporated herein by reference), the yeast transcriptional corepressor Ure2p (uretupamines) (Kuruvilla et al. Dissecting glucose signalling with diversity-oriented synthesis and small-molecule microarrays. Nature 2002; 416(6881):653-7; incorporated herein by reference), and the Hap3p subunit of the yeast HAP transcription factor complex (haptamides) (Koehler et al. Discovery of an inhibitor of a transcription factor using small molecule microarrays and diversity-oriented synthesis. J. Am. Chem. Soc. 2003; 125(28):8420-1; incorporated herein by reference). Each of these screens involved SMMs in which only one DOS library was contained on a given slide. More recently, we sought to prepare an SMM that contains sub-libraries from various DOS synthetic routes in one array. The goal of preparing such an SMM is to allow researchers to sample the various sublibraries in one array and then prioritize screens of the full DOS libraries based on the initial screening results from the diverse subset. In this Example we report the use of isocyanate-functionalized glass slides to make a small-molecule “diversity microarray” containing several collections of DOS compounds coming from various solid-phase organic synthesis routes (Burke et al. Generating diverse skeletons of small molecules combinatorially. Science 2003; 302(5645):613-8; Burke et al. A synthesis strategy yielding skeletally diverse small molecules combinatorially. J. Am. Chem. Soc. 2004; 126(43):14095-104; Chen et al. Convergent diversity-oriented synthesis of small-molecule hybrids. Angew Chem Int Ed Engl 2005; 44(15):2249-52; Kumar et al. Small-molecule diversity using a skeletal transformation strategy. Org Lett 2005; 7(13):2535-8; Lo et al. A library of spirooxindoles based on a stereoselective three-component coupling reaction. J. Am. Chem. Soc. 2004; 126(49):16077-86; Stavenger et al. Asymmetric Catalysis in Diversity-Oriented Organic Synthesis: Enantioselective Synthesis of 4320 Encoded and Spatially Segregated Dihydropyrancarboxamides. Angew Chem Int Ed Engl 2001; 40(18):3417-3421; Wong et al. Modular synthesis and preliminary biological evaluation of stereochemic ally diverse 1,3-dioxanes. Chem Biol 2004; 11(9):1279-91; each of which is incorporated herein by reference) and commercially available bioactive compounds, including natural products, on the same slide (FIG. 1).

Prior strategies aimed at ligand discovery using SMMs have relied on incubation with a purified protein of interest. Potential applications of these protocols have been limited by challenges in protein biochemistry involving expression of large proteins, solubility, post-translational modification state, activity and yield. Furthermore, without commercial availability of a protein target of interest, investigators without expertise in protein biochemistry may be limited in their capacity to screen SMMs. Here, we describe the optimization of a robust, efficient SMM screening methodology which allows the detection of specific protein-small molecule interactions using epitope-tagged target proteins directly from cell lysates without purification. We demonstrate that the new attachment chemistry is compatible with detection of known interactions between various small molecules and FKBP12 (Harding et al. A receptor for the immunosuppressant FK506 is a cis-trans peptidyl-prolyl isomerase. Nature 1989; 341(6244):758-60; Siekierka et al. A cytosolic binding protein for the immunosuppressant FK506 has peptidyl-prolyl isomerase activity but is distinct from cyclophilin. Nature 1989; 341(6244):755-7; each of which is incorporated herein by reference) obtained directly from cellular lysates. Previous research reporting the detection of specific interactions using complex lysates have typically involved the addition of known, purified proteins (Reddy et al. Protein “fingerprinting” in complex mixtures with peptoid microarrays. Proc Natl Acad Sci USA 2005; 102(36):12672-7; incorporated herein by reference) or has required incubation in solution with focused libraries of covalent probes conjugated to nucleic acids prior to spatial arraying on an oligonucleotide array (Winssinger et al. PNA-encoded protease substrate microarrays. Chem Biol 2004; 11(10):1351-60; Winssinger et al. Profiling protein function with small molecule microarrays. Proc Natl Acad Sci USA 2002; 99(17):11139-44; each of which is incorporated herein by reference). The ability to detect selective interactions in cellular lysates without protein purification is appealing for ligand discovery, target identification, antibody and protein specificity profiling, as well as for future applications such as signature discovery for cellular states and diagnostic tool development.

### Results

Small molecules containing nucleophiles with a range of reactivities were arrayed onto a weakly electrophilic surface that reacts slowly with either the small molecules or ambient moisture and yields no potentially deleterious byproducts such as an acid. As shown in FIG. 2, γ-aminopropylsilane slides (S1) were coated with a short polyethylene glycol (PEG) spacer and coupled to 1,6-diisocyanatohexane via a urea bond to generate putative isocyanate-functionalized glass slides (S2). Slides printed with compound stock solutions were then placed in a dry environment and exposed to a pyridine vapor that catalyzes the covalent capture of small molecules onto the slide surface (S3).

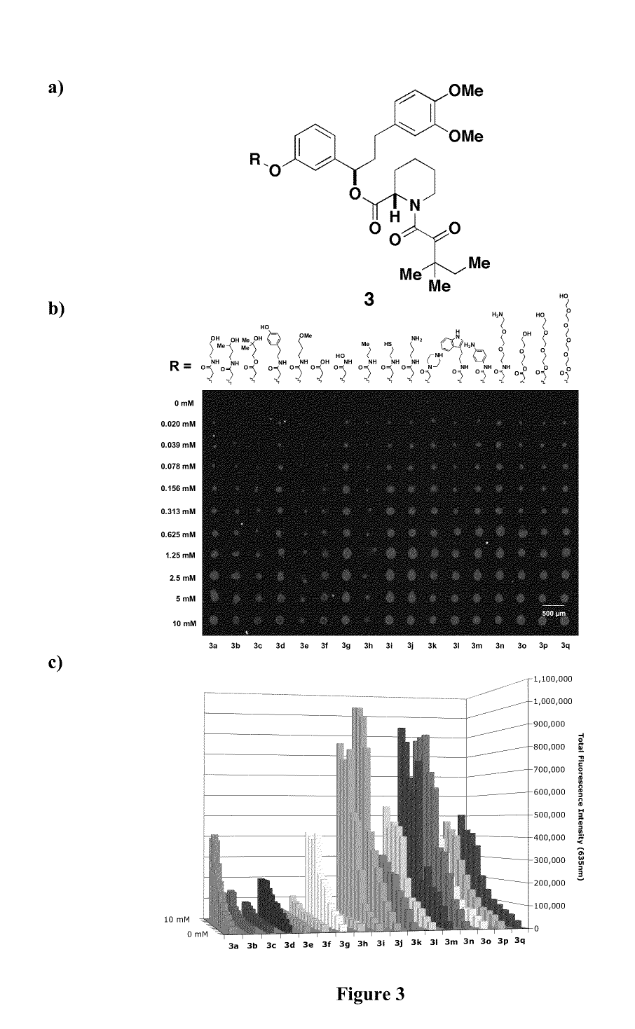

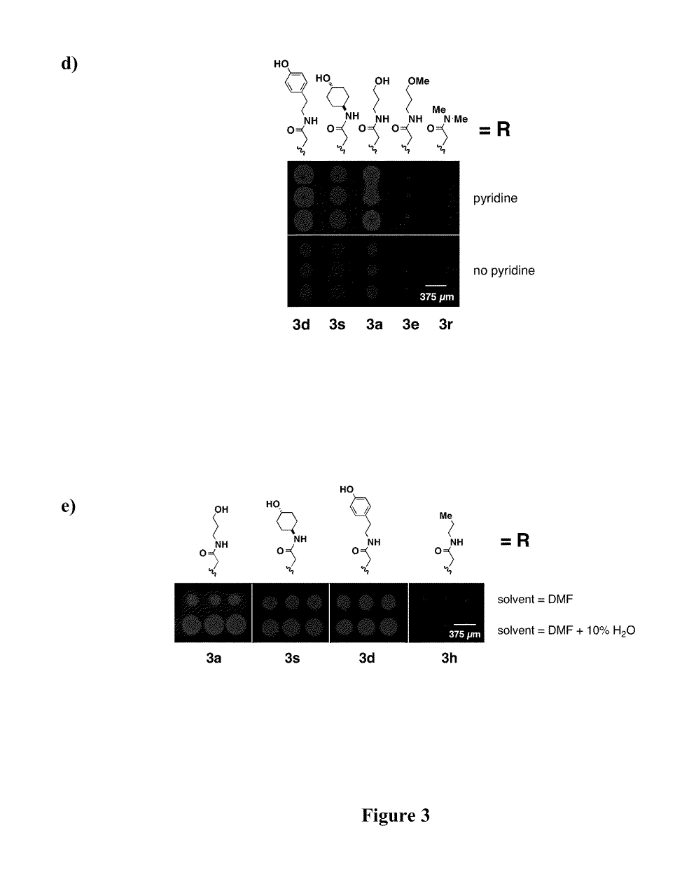

To evaluate this approach, a robotic microarrayer was used to print a series of synthetic FKBP12 ligands (Holt et al. Design, synthesis and kinetic evaluation of high-affinity FKBP ligands and the X-Ray crystal structures of their complexes with FKBP12. J Am Chem Soc 1993; 115:9925-9938; incorporated herein by reference) that were derivatized so as to present a primary alcohol (3a, 3o, 3p, 3q), secondary alcohol (3b), tertiary alcohol (3c), phenol (3d), methyl ether (3e), carboxylic acid (3f), hydroxamic acid (3g), methyl (3h), thiol (3i), primary amine (3j, 3n), secondary amine (3k), indole (3l), or aryl amine (3m) onto the isocyanate-derivatized slides (FIG. 3a,b). The site of modification for each FKBP12 ligand has previously been shown to be tolerant to substitution as 3 is a parent structure for chemical inducers of dimerization (Keenan et al. Synthesis and activity of bivalent FKBP12 ligands for the regulated dimerization of proteins. Bioorg Med Chem 1998; 6(8):1309-35; incorporated herein by reference). The ligands were printed in serial two-fold dilutions (10 mM to 20 μM) using DMF as a solvent. The printed slides were exposed to pyridine vapor, quenched with ethylene glycol, and washed extensively with DMF, THF, and methanol. Dried slides were probed with FKBP12-GST (Harding et al. A receptor for the immunosuppressant FK506 is a cis-trans peptidyl-prolyl isomerase. Nature 1989; 341(6244):758-60; Siekierka et al. A cytosolic binding protein for the immunosuppressant FK506 has peptidyl-prolyl isomerase activity but is distinct from cyclophilin. Nature 1989; 341(6244):755-7; each of which is incorporated herein by reference), followed by a Cy5™-labeled anti-GST antibody, and scanned for fluorescence at 635 nm using GenePix Pro 6.0 software (Molecular Devices, Union City, Calif.). As shown in FIG. 3, the intensity of fluorescent signals corresponding to FKBP12-GST varied according to both the functional group presented for attachment and concentration of ligand. Feature diameter was dependent on the concentration of ligand and at higher concentrations the average diameter was 250 μm. The primary amines, aryl amine, and thiol appear to have the highest immobilization levels. Fluorescence intensities for the primary alcohols, phenol, hydroxamic acid, secondary amine, and indole are also consistent with significant immobilization. The secondary alcohol, carboxylic acid, and tertiary alcohol were immobilized in lower amounts. At 1.25 mM, a typical concentration for our compound stock solutions, trace levels of primary amides 3e and 3h were detected whereas the N,N-substituted amide 3r (FIG. 3d) was not. The addition of polyethylene glycol spacers of varying lengths to the ligand (3n-3q) did not make a significant impact on the feature morphology or fluorescence intensity when probed with purified protein. Additionally, polyethylene glycol spacers of varying lengths (n=0, 2, 4, 8, 70) were added to surface S2 and compared (data not shown). Surfaces with shorter PEG chains (n=2, 4, 8) were equivalent and displayed improved signal-noise ratios over the surface without PEG. The surface with longer PEG chains displayed the lowest fluorescence levels in the binding assay and gave inconsistent spot morphologies.

Fluorescence levels were significantly reduced when pyridine vapor was omitted from the procedure (FIG. 3d) Immobilization levels were slightly enhanced when the slides were exposed to pyridine at 37° C. (data not shown). To test the sensitivity of this capture method to moisture present in the compound stock solutions used for printing, 1 mM solutions of FKBP12 ligands 3a, 3b, 3c, and 3e in 9:1 DMF:ddH₂O were arrayed in triplicate onto isocyanate-derivatized slides (FIG. 3e). Fluorescence intensities were equivalent with those of compounds printed directly from DMF. Tolerance to water is an important consideration for SMM preparation because compound stock solutions in DMF and DMSO appear to take on water over time as they move in and out of freezer storage (Cheng et al. Studies on repository compound stability in DMSO under various conditions. J. Biomol. Screen 2003; 8(3):292-304; incorporated herein by reference). Small molecules printed from DMSO were also captured using this method with smaller feature diameters (100-150 μm) than compounds printed from DMF (˜250-300 μm).

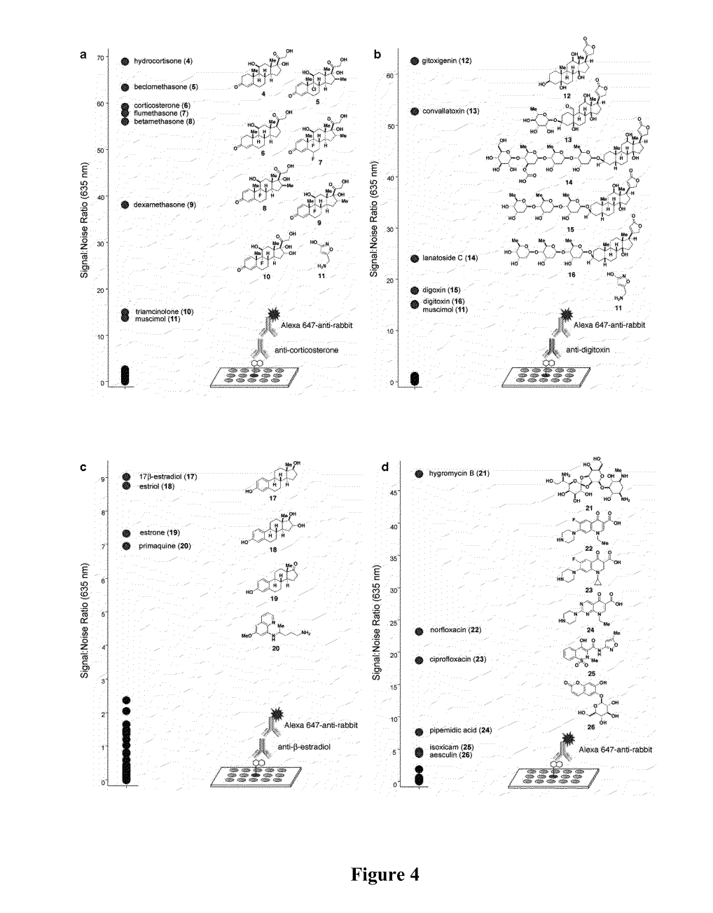

To investigate the suitability of our approach for printing compounds that have not been intentionally synthesized with appendages for covalent capture, more than 300 commercially available bioactive compounds were printed onto isocyanate-functionalized slides. We screened these bioactive microarrays using rabbit primary antibodies against corticosterone, digitoxin, and 17β-estradiol, followed by a fluor-labeled goat anti-rabbit secondary antibody. The signal-to-noise ratio (SNR) was determined by calculating intensity at 635 nm and adjusting for local background for each feature on replicate arrays, and data were compared to replicate control arrays incubated with the labeled secondary antibody alone (FIG. 4). Six bioactives, with signal-to-noise ratios >3.0, were found in replicate arrays to bind to the labeled polyclonal secondary antibody alone. None of the compounds were autofluorescent at 635 nm as judged by arrays probed with PBS buffer alone (data not shown). Hygromycin B, an aminoglycoside antibiotic, gave the highest adjusted signal-to-noise ratio (mean SNR 47.6). Three quinolone antibiotics, norfloxacin, ciprofloxacin, and pipemidic acid displayed mean adjusted fluorescent intensities greater than 3.0 in at least one experiment. In the anti-corticosterone antibody binding profile, hydrocortisone (mean SNR 68.9), beclomethasone (63.3), and corticosterone (59.2), corticosteroids related in structure, scored as positives. Gitoxigenin (mean SNR 62.5), convallatoxin (52.7), lanatoside C (24.0), digoxin (17.8) and digitoxin (15.1), all cardioactive steroid glycosides, likewise scored as positives in replicate anti-digitoxin antibody experiments. 17β-estradiol (mean SNR 9.0), estriol (8.7) and estrone (7.3), primary estrogenic hormones varying in the number of reactive groups for capture, scored as positives in the anti-17β-estradiol binding profile. The antibody-binding profiles demonstrate that small molecules with multiple nucleophilic functional groups can be printed and detected using isocyanate-mediated capture. Additionally, these data demonstrate a facile approach for profiling the specificity of immunoglobulins for small molecules.

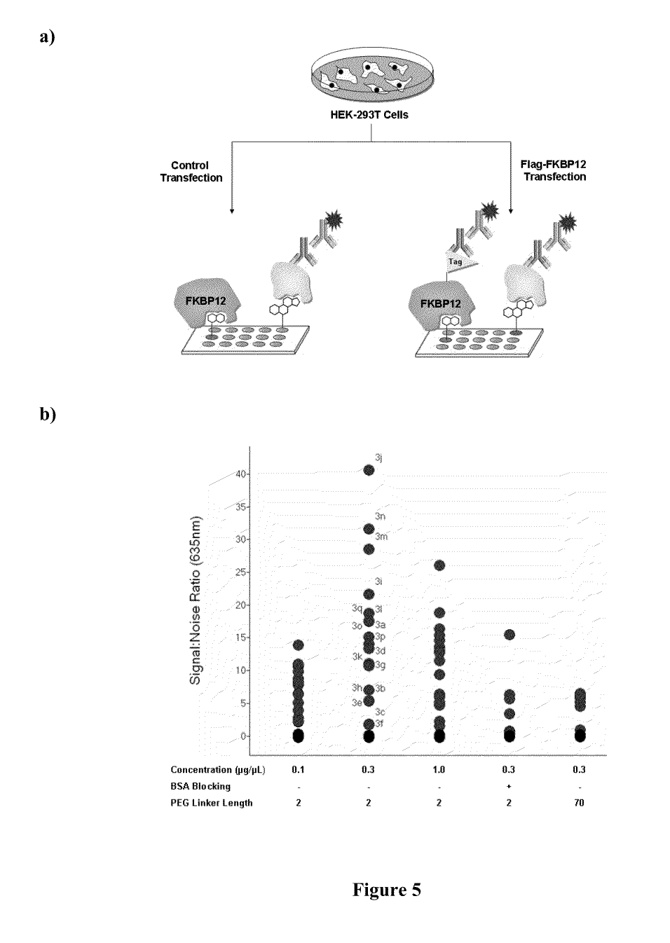

We aimed to expand the scope of this method to include the detection of interactions between small molecules and target proteins expressed in mammalian cells without prior purification. Toward this end, a screening protocol was developed whereby SMMs incubated with cellular lysates bearing over-expressed epitope-tagged proteins of interest are compared with control SMMs incubated with mock-transfected cellular lysates (FIG. 5a). Following mild lysis and clarification by centrifugation, cellular lysates were incubated on SMMs. Subsequently, the arrays were serially incubated with a primary anti-epitope antibody, and a Cy5™-conjugated secondary antibody. A brief wash with PBST and mild agitation followed each incubation. Fluorescence intensity was detected and SNR was calculated, compared and averaged for corresponding features on replicate arrays.

We explored this approach by screening the array of AP1497 derivatives (as in FIG. 3b) against HEK-293T lysates prepared from mammalian cells transiently transfected with a construct engineered to over-express FLAG-FKBP12. Optimization experiments were undertaken with a step-wise introduction of variation to identify parameters maximizing protocol robustness. Arrays were derived from the same printing series, and were scanned for fluorescence using identical laser power and photomultiplier tube gain. Experimental variables were compared using mean SNR for ligands arrayed at a uniform, standard concentration of 1.25 mM, as depicted in FIG. 5b. To determine whether the total protein concentration affects ligand detection, SMMs were incubated with lysates varying in concentration from 0.1 to 1.0 ug/uL. Maximum fluorescence intensity and SNR for each feature proved optimal at 0.3 ug/uL. Blocking incubations are commonly employed in protocols involving SMMs. Given the complex milieu of cellular lysates, we were interested in exploring whether blocking prior to sample incubation is required. Blocking with BSA was found to diminish both the maximum signal intensity and background adjusted signal (SNR) when incubating SMMs with cellular lysates. Interactions between printed ligands and macromolecules may be enhanced with the introduction of a polymeric polyethylene glycol (PEG) spacer. Nonspecific background interactions may also be minimized with a slide surface coated with PEG. To investigate the effect of spacer length on fluorescent detection and SNR, PEG spacer length was varied in printed AP1497 derivative SMMs. A marked decrease in the SNR was observed for each printed feature with a long (n˜70) PEG spacer compared to a substantially shorter spacer (n=2). Additional optimization experiments and the detailed, optimized screening protocol for SMMs using cellular lysates are presented below.

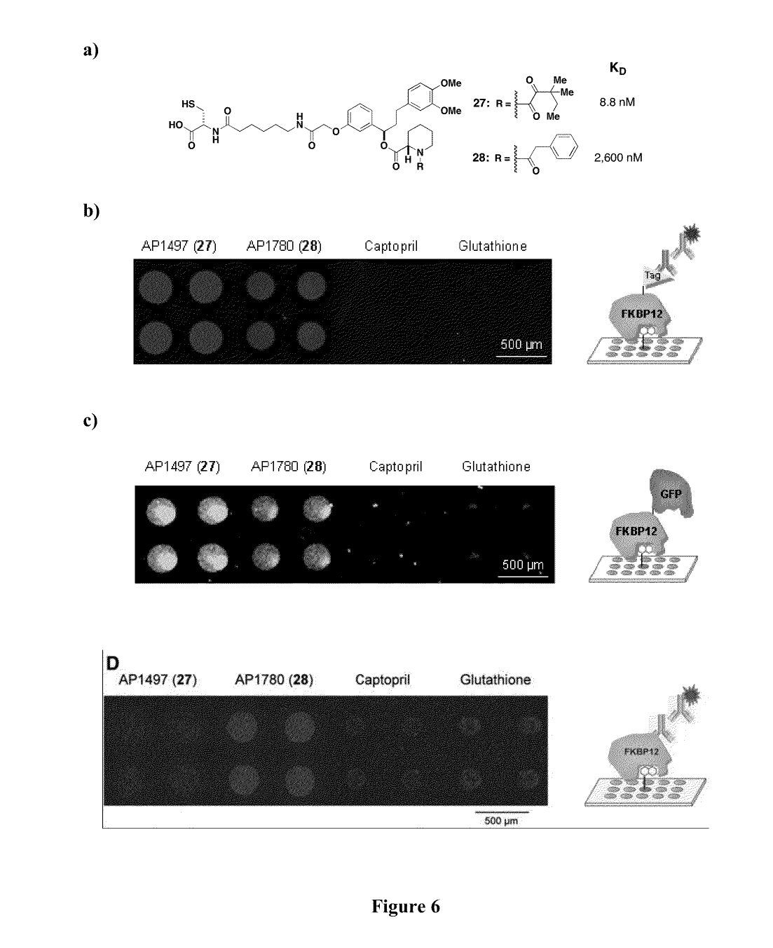

Recognizing the high affinity of AP1497 for FKBP12 (K_{D}=8.8 nM), we were interested in assessing the ability of this technique to identify lower affinity interactions as may be detected in screening experiments. Using the isocyanate capture method, focused arrays of two ligands with disparate affinity for FKBP12 (FIG. 6A) were printed with control bioactives. The optimized screening protocol allowed the specific identification of ligands with K_{D} as a high as 2.6 μM (FIG. 6B) (MacBeath et al. Printing proteins as microarrays for high-throughput function determination. Science 2000; 289(5485):1760-3; incorporated herein by reference). To determine whether this method would allow the detection of low affinity interactions between small molecules and chimeric fluorescent proteins, SMMs were incubated with lysates from mammalian cells transiently transfected with a vector encoding an EGFP-FKBP12 fusion protein. Incubated slides were washed briefly with PBST and scanned for fluorescence at 488 nm. Identification of ligands with low binding affinity was observed without the requirement of primary and fluorescently labeled secondary antibodies (FIG. 6C). Transient transfection of cells in tissue culture with protein expression constructs typically results in protein overexpression, as in the experiments above. In the context of ligand discovery, this may prove desirable; however, additional applications of SMMs such as profiling of cellular states involves the detection of specific interactions with endogenously expressed proteins by using target protein-specific antibodies. To explore this possibility, SMMs were incubated with lysates from untransfected 293T cells. Subsequent incubation with a commercially available polyclonal antibody against the N-terminal region of FKBP12 and secondary fluorophore-conjugated antibody allowed the detection of specific interactions between endogenous FKBP12 and ligands with K_{D} as high as 2.6 μM (FIG. 6D).

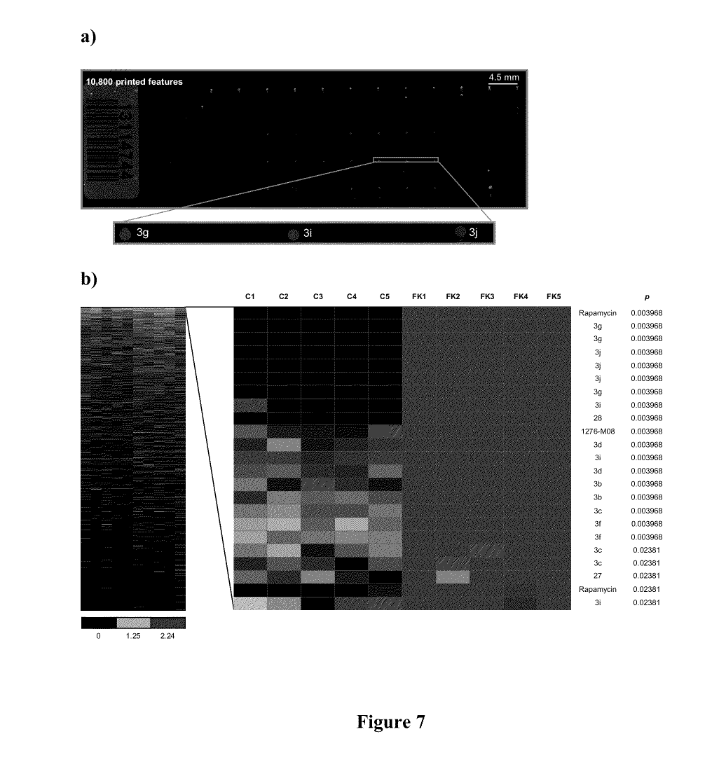

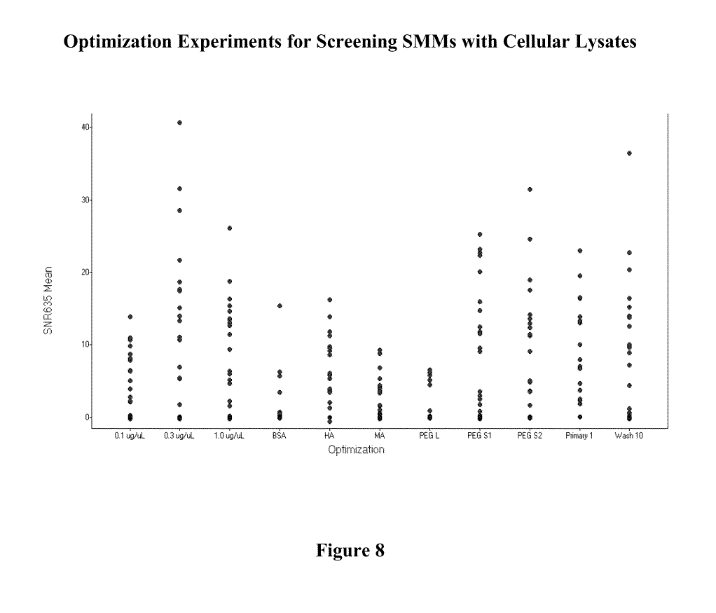

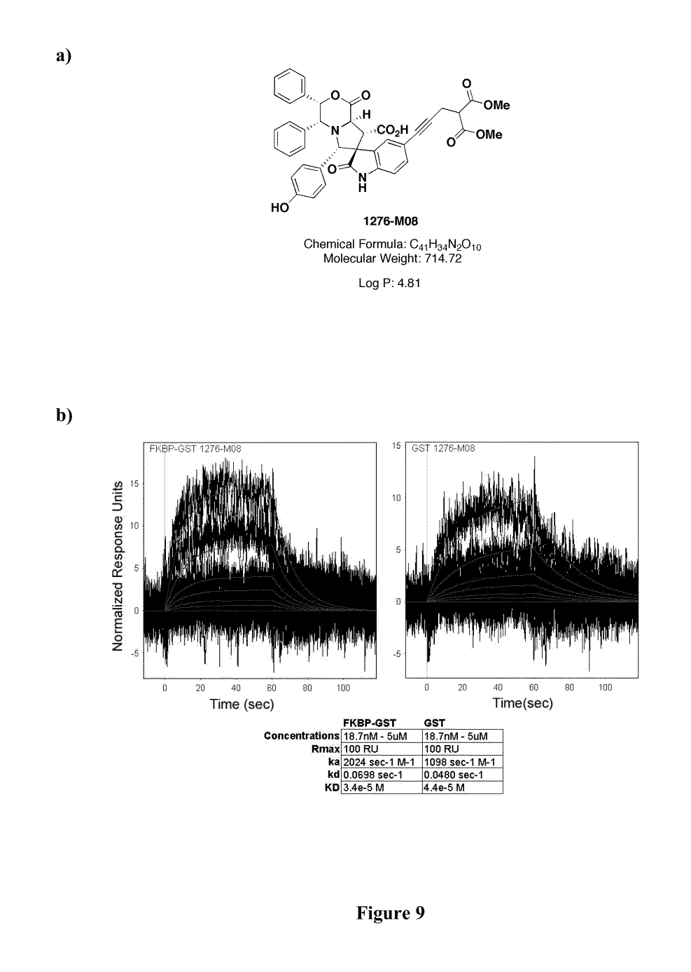

To investigate the robustness of our optimized lysate protocol as a screening methodology, a diverse SMM was printed containing 10,000 bioactive small molecules, natural products and small molecules originating from diversity-oriented syntheses. The microarray also included twenty-seven features corresponding to synthetic ligands to FKBP12 (3-5), and the immunosuppressant and anticancer natural product rapamycin, a known ligand to FKBP12. Ten cellular lysates (five control and five Flag-FKBP12) were independently prepared and incubated with a diversity SMM. After incubation with primary and Cy5-labeled secondary antibodies, slides were scanned for fluorescence at 635 nm and local background correction (SNR) was calculated. Among five replicate SMMs with Flag-FKBP12-expressing lysate, all twenty-seven printed ligands to FKBP12, including rapamycin and the low affinity synthetic ligand 5, were detected. A representative array is presented in FIG. 7a.

To interrogate statistically the ability of our technique to identify ligands to a protein of interest on a diverse array, locally corrected feature intensity (SNR635) was dichotomized by a threshold intensity of 2.24, established by the maximal SNR intensity of arrayed solvent. Features with SNR intensities greater than 2.24 were classified as positives. Features from control- or Flag-FKBP12-incubated arrays were compared using Fisher's Exact test, and contingency tables were generated for 104 solvent-only features which appeared as hits in at least one experiment. At a significance level of 0.05, twenty-four cells were found to have a significant p-value (FIG. 7b). One DOS compound, 1276-M08, also scored as an assay positive. Binding was confirmed by surface plasmon resonance, however the resynthesized, major product from the well was found to bind both GST and GST-FKBP12. by surface plasmon resonance indicating that the molecule is likely not a selective ligand for FKBP12.

### Discussion

We used a covalent-capture strategy for small molecules that makes use of a well-characterized chemical reaction (Vandenabeele-Trambouze et al. Reactivity of organic isocyanates with nucleophilic compounds: amines, alcohols, thiols, oximes, and phenols in dilute organic solutions. Advanced Environmental Research 2001; 6:45-55; Ameringer et al. Ultrathin functional star PEG coatings for DNA microarrays. Biomacromolecules 2005; 6(4):1819-23; Chun et al. Diisocyanates as novel molecular binders for monolayer assembly of zeolite crystals on glass. Chem Commun (Camb) 2002(17):1846-7; Guo et al. Direct fluorescence analysis of genetic polymorphisms by hybridization with oligonucleotide arrays on glass supports. Nucleic Acids Res 1994; 22(24):5456-65; Sompuram et al. A water-stable protected isocyanate glass array substrate. Anal Biochem 2004; 326(1):55-68; each of which is incorporated herein by reference) and allows preparation for the first time of microarrays containing small molecules coming from both natural and synthetic sources. The isocyanate-mediated capture is applicable to compounds containing a variety of nucleophilic functional groups and does not require compounds to contain a special reactive appendage, such as an alcohol or azide (Barnes-Seeman et al. Expanding the functional group compatibility of small-molecule microarrays: discovery of novel calmodulin ligands. Angew Chem Int Ed Engl 2003; 42(21):2376-9; Hergenrother et al. Small molecule microarrays: covalent attachment and screening of alcohol-containing small molecules on glass slides. J. Am. Chem. Soc. 2000; 122:7849-50; Kohn et al. Staudinger ligation: a new immobilization strategy for the preparation of small-molecule arrays. Angew Chem Int Ed Engl 2003; 42(47):5830-4; each of which is incorporated herein by reference), to be introduced during synthesis for covalent capture in the array. The isocyanate functionality generates no byproducts; in contrast to several previous capture agents, including those using electropositive chlorine moieties. The latter result in the deposition and concentration of an acidic residue in the vicinity of the small molecule, which could result in partial degradation of the small molecule and obfuscation of the screening results. Compounds containing multiple nucleophilic functional groups also have the potential to be displayed in varying orientations in a given spot. Multiple modes of display may allow proteins to sample different binding orientations in a given microarray feature. The isocyanate slides may, however, react with a nucleophile that is required for protein binding and may therefore lead to some false negatives in screens. Due to the potential heterogeneity within printed features, we prefer to use data coming from surface plasmon resonance-based secondary binding assays rather than microarray fluorescence intensity to prioritize positives for follow-up. This approach allows us to identify rapidly candidate ligands using the high-throughput microarray screening platform and the surface plasmon resonance platform to characterize positives in real-time and quantitative assays.

The capture method has allowed us to produce microarrays that contain compounds derived from a variety of solid-phase syntheses alongside natural products and bioactive compounds, such as FDA-approved drugs. These arrays contain greater chemical diversity and therefore are more desirable for screening against larger panels of proteins. In our experience, researchers with one protein of interest often prefer to screen multiple microarrays containing compounds from individual syntheses but begin by screening the diversity array to help guide their choices about which libraries to screen further.

In an effort to verify the printing of complex collections of small molecules with variable functional groups, we probed a diverse SMM with a series of antibodies with known specificities for bioactive small molecules. Structural analogs of the known target of these antibodies were also identified, indicating that large, diverse collections of printed molecules may yield insights into structure-binding properties of immunoglobulins. This approach has implications for immunoglobulin profiling as has been reported previously using focused carbohydrate arrays (Wang D, Liu S, Trummer B J, Deng C, Wang A. Carbohydrate microarrays for the recognition of cross-reactive molecular markers of microbes and host cells. Nat Biotechnol 2002; 20(3):275-81; incorporated herein by reference). Importantly, profiling antibody specificity among large, diverse libraries of small molecules as presented herein offers unique opportunities for rapid diagnostic, therapeutic, neutralizing, and catalytic antibody discovery.

SMMs resulting from isocyanate-mediated capture are also compatible with binding screens involving total cell lysates containing overexpressed, epitope-tagged proteins in cell lysate. The ability to screen directly from lysates saves substantial time and effort by avoiding protein purification. This lysate methodology offers the possibility of ligand discovery for proteins which have eluded comprehensive approaches at purification. Lysate screens are more biologically relevant as some proteins of interest may reside within protein complexes or require a protein partner to remain active. Proteins obtained directly from mammalian cellular lysates are also more likely to fold properly and possess post-translational modifications associated with an active or desirable tertiary structure. The proteins from lysate may also serve to block the surface thereby creating a competitive assay. The linkage of the small molecule to the surface prepared using isocyanate-capture also appears to be stable to cellular esterases and proteases under lysate screening conditions as the slides can be stripped under denaturing conditions and reprobed (data not shown). Signal-to-noise ratios in lysate experiments using isocyanate capture are improved over surfaces we have prepared that involve linkage to the surface through an ester bond. Consequently, we believe this new capability constitutes a major advance in the SMM method and should expand its use as a method to discover small-molecule partners for proteins of interest. The diversity of printed features and the compatibility of the SMM surface with this lysate screening protocol also allow profiling of complex mixtures of proteins derived from cellular lysates without prior purification. A detailed study of lysate applications on SMMs is underway in our laboratories.

More than a thousand replicate diversity SMMs have been printed to date. Through collaborations involving several laboratories, more than fifty proteins, including single purified proteins, purified protein complexes, and proteins from clarified cell lysates, have been screened against these microarrays. Of more than 100 interactions tested, 86% retest as binders with estimated dissociation constants of 0.5-μM in a secondary surface plasmon resonance-based assay that involves immobilization of the target protein on a dextran-coated sensor surface and injection of the compound at varying concentrations (Barnes-Seeman et al. Expanding the functional group compatibility of small-molecule microarrays: discovery of novel calmodulin ligands. Angew Chem Int Ed Engl 2003; 42(21):2376-9; incorporated herein by reference). Compounds that do not retest are typically classified as insoluble, nonspecific binders to dextran, or false-positives.

In summary, we have developed a new method for preparing small-molecule microarrays that can be applied to compounds containing a range of nucleophilic functional groups thereby increasing both the diversity and quantity of compounds, from natural or synthetic sources, that can be immobilized for microarray-based binding screens. We were able to detect and confirm the presence of selected printed small molecules, and structurally related compounds, using antibodies. Finally, we used this chemistry to prepare diversity SMMs containing nearly 10,000 small molecules and used the microarrays to demonstrate that the surface is compatible with detection of interactions using total protein from cellular lysates without any purification. Future efforts will make use of antibodies and the lysate-compatible diversity SMMs for profiling binding selectivity and changes in cell state using small-molecule binding as a signature.

### Experimental Procedures

Materials.

Bioactive small molecules and natural products were purchased from commercial sources. DOS molecules were obtained from the Broad Chemical Biology Program. Compound 3s was the gift of Dr. John Tallarico. Compounds 27, 28 were obtained from Dr. Timothy Clackson of Ariad Pharmaceuticals. The Flag-FKBP12 mammalian expression construct was the gift of Dr. Paul Clemons. The EGFP-FKBP12 mammalian expression vector was constructed using the Creator™ cloning system purchased from Clontech Laboratories and an FKBP12 library vector obtained from the Harvard Institute of Proteomics. Antibodies against corticosterone, estradiol, and digitoxin were purchased from Sigma. Mouse Anti-Flag™ monoclonal antibody was purchased from Sigma. Alexa Fluor® 647 goat-anti-rabbit antibody was purchased from Invitrogen. Cy5™-labeled goat anti-GST and rabbit anti-mouse antibodies were purchased from Amersham Biosciences. Slides were scanned either using an Axon 4000B scanner at 5 μm resolution using 635 nm and 532 nm lasers or using an Axon 4200A scanner at 5 μm resolution using 488 nm and 532 nm lasers. Arrays were analyzed using GenePix Pro 6.0 software purchased from Molecular Devices.

General Methods.

All commercially available materials were used without further purification. All reaction solvents except DMF were dispensed from a solvent purification system wherein solvents are passed through packed activated alumina column DMF was Aldrich anhydrous grade. Solvents for other uses were commercially available HPLC grade purchased from Fisher. All reactions were carried out in oven dried standard laboratory glassware under positive Argon pressure. Reactions were monitored by thin layer chromatography using Merck silica gel 60 F254 plates. Compounds were visualized by UV (254 nm) or phosphomolybdic acid. Flash column chromatography was performed using Merck silica gel 60 (230-400 mesh). All NMR spectra were recorded on a Varian Inova AS500 spectrometer. Chemical shifts are expressed in ppm relative to residual solvent signals. LC-MS was performed on a Waters Alliance 2690 HPLC system with a Waters Symmetry C18 column. Compounds were detected by a Waters 996 photo diode array detector and a Micromass LCZ (ESI) spectrometer. CH₃CN and 0.1% formic acid in water were used as solvents. The ratio was 15% CH₃CN/85% water at 0 min and 100% CH₃CN at 5 min with linear gradient followed by 1 min of 100% CH₃CN. Preparative HPLC was performed on Waters Delta 600 with 2487 Dual Wavelength detector using a Symmetry C18 semi-preparative column and acetonitrile (0.1% trifluoroacetic acid)/water (0.1% trifluoroacetic acid) as mobile phase.

General Protocol for Isocyanate Slide Preparation.

Amino-functionalized glass slides, either prepared as described previously (MacBeath G, Koehler A N, Schreiber S L. Printing small molecules as microarrays and detecting protein-small molecule interactions en masse. J Am Chem Soc 1999; 121:7967-68; incorporated herein by reference) or commercially available γ-aminopropylsilane GAPS™ slides (Corning), were incubated in a solution of Fmoc-8-amino-3,6-dioxaoctanoic acid (10 mM, Neosystem), PyBOP (10 mM), and iPr₂NEt (20 mM) in DMF for at least 4 h. The slides were washed in DMF to remove excess coupling solution and incubated in a solution of 10% (v/v) piperidine in DMF for 30 min (room temperature) to remove the Fmoc group from the surface. Following a rinse in DMF, the slides were activated in a solution of 10% (v/v) 1,6-diisocyanatohexane (Aldrich) in DMF for 30 min at room temperature. Three brief rinses in THF allow for complete removal of the activating solution and fast drying of the slides before placement on the robotic microarrayer platform. Depending on the length of the printing process, printed slides were allowed to dry for at least 10 min (print runs of >2 h) and up to 2 h (short print runs) before they were placed into metal racks in a glass vacuum desiccator. A three-way adapter was attached to the desiccator, with tubing leading to a vacuum line and a round-bottom flask containing approximately 1 mL of pyridine. Once the desiccator and flask were fully evacuated, the vacuum line was shut off and the catalytic pyridine vapor normalized the pressure for at least 4 h. The slides were then immersed in a solution of ethylene glycol (1 M in DMF) and 1% (v/v) pyridine for 10 min to quench the surface. The slides were washed twice in DMF for 30 min, washed once in ethanol for 30 min, dried by centrifugation, and stored at −20° C. prior to screening. Slides were stored up to 6 months using these conditions.

Diversity Small-Molecule Microarray Preparation.

Small molecules from the diversity set were arrayed onto isocyanate-functionalized glass slides using an OmniGrid® 100 Microarrayer (Genomic Solutions) outfitted with a ArrayIt™ Stealth 48-pin printhead and SMP3 spotting pins (TeleChem International, Inc.) as described previously (Barnes-Seeman et al. Expanding the functional group compatibility of small-molecule microarrays: discovery of novel calmodulin ligands. Angew Chem Int Ed Engl 2003; 42(21):2376-9; incorporated herein by reference). The microarrays contain 10,800 printed features with 48 subarrays of 15×15 features with 320 μm center-to-center spacing. Solutions of small molecules (˜1 mM in DMF) were printed from 384-well polypropylene plates (Abgene). Twenty-eight plates containing 9,152 DOS compounds (Burke et al. Generating diverse skeletons of small molecules combinatorially. Science 2003; 302(5645):613-8; Burke et al. A synthesis strategy yielding skeletally diverse small molecules combinatorially. J Am Chem Soc 2004; 126(43):14095-104; Chen et al. Convergent diversity-oriented synthesis of small-molecule hybrids. Angew Chem Int Ed Engl 2005; 44(15):2249-52; Kumar et al. Small-molecule diversity using a skeletal transformation strategy. Org Lett 2005; 7(13):2535-8; Lo et al. A library of spirooxindoles based on a stereoselective three-component coupling reaction. J. Am. Chem. Soc. 2004; 126(49):16077-86; Stavenger et al. Asymmetric Catalysis in Diversity-Oriented Organic Synthesis Enantioselective Synthesis of 4320 Encoded and Spatially Segregated Dihydropyrancarboxamides. Angew Chem Int Ed Engl 2001; 40(18):3417-3421; Wong et al. Modular synthesis and preliminary biological evaluation of stereochemically diverse 1,3-dioxanes. Chem Biol 2004; 11(9):1279-91; each of which is incorporated herein by reference), 336 bioactives, 72 control compounds, and 1,192 blank wells containing DMF were printed. Forty-eight wells of a twenty-ninth plate, containing various concentrations of rhodamine derivatives (˜1 mM, DMF) (MacBeath et al. Printing small molecules as microarrays and detecting protein-small molecule interactions en masse. J Am Chem Soc 1999; 121:7967-68; incorporated herein by reference), were printed in the final dip to serve as fluorescent markers on the array that frame the subarrays. Each pin was washed three times for five seconds in acetonitrile and vacuum-dried for three seconds between picking up samples from the wells in an effort to minimize carryover contamination of samples. One hundred arrays were printed in a given print run and more than 1,000 copies of the diversity microarray have been printed to date. Quality control for each print run involved scanning arrays prior to screening and looking for the presence or absence of various fluor control features as well as screens to detect selected known protein-ligand interactions.

Microarray Screens with Purified FKBP12-GST.

Microarrays were incubated with 300 μL of a 1 μg/mL solution of purified FKBP12-GST (Harding et al. A receptor for the immunosuppressant FK506 is a cis-trans peptidyl-prolyl isomerase. Nature 1989; 341(6244):758-60; Siekierka et al. A cytosolic binding protein for the immunosuppressant FK506 has peptidyl-prolyl isomerase activity but is distinct from cyclophilin. Nature 1989; 341(6244):755-7; each of which is incorporated herein by reference) in PBST buffer for 30 min at room temperature. The arrays were briefly rinsed with PBST and then washed twice in PBST (1 min for each wash) on an orbital platform shaker. Arrays were then incubated with 300 μL of a 0.5 μg/mL solution of Cy5™-labeled goat anti-GST antibody in PBST for 30 min at room temperature. Probed arrays were rinsed in PBST, washed three times in PBST (2 min for each wash), and washed once in PBS (2 min). Arrays were dried by centrifugation and scanned for fluorescence at 635 nm on a Genepix 4000B microarray scanner. Control arrays were probed with the secondary labeled antibody, the primary antibody followed by labeled secondary antibody, and GST followed by both primary and secondary antibodies to ensure that fluorescent signals were due to binding of FKBP12 to the printed ligands. To analyze the array features containing ligands 3a-3q (FIG. 3b), total fluorescence intensity values were calculated for a set 300 μm diameter centered over each feature using GenePix Pro 6.0 software. Intensities for each ligand at varying concentrations are displayed in a graph (FIG. 3c).

Small-Molecule Microarray Profiles with Antibodies Against Natural Products.

Microarrays printed with natural products and bioactives were incubated with various antibodies to detect specific compounds. In the first incubation step, arrays were incubated with 300 μL of one of the following: PBST buffer (control), a 1:500 solution of rabbit anti-corticosterone whole antiserum in PBST, 1:500 solution of rabbit anti-17β-estradiol whole antiserum in PB ST for 30 mM at room temperature. The arrays were briefly rinsed with PBST and then washed twice in PB ST. All arrays were then incubated with 300 μL of a 1:1000 solution of Alexa Fluor® 647 goat-anti-mouse polyclonal secondary antibody in PBST for 30 mM at room temperature. Probed arrays were rinsed in PBST, washed three times in PBST, and washed once in PBS. Arrays were dried by centrifugation and scanned for fluorescence at 635 nm Signal-to-noise ratio was calculated for each feature using adjusted diameters.

Diversity Microarray Screens with Flag-FKBP12 from Lysates.

Routine culture of HEK-293T cells was performed in DMEM supplemented with penicillin/streptomycin and 10% fetal bovine serum (FBS). Transfection of HEK-293T cells with a mammalian overexpression vector bearing a Flag™ epitope-tagged FKBP12 coding sequence was performed by FuGENE6 lipid transfection (Roche Applied Science). Cells were harvested after 48 h, and clarified lysates were prepared by incubation with MIPP lysis buffer (20 mM NaH₂PO₄, pH 7.2, 1 mM Na₃VO₄, 5 mM NaF, 25 mM β-glycerophosphate, 2 mM EGTA, 2 mM EDTA, 1 mM DTT, 0.5% (v/v) Triton X-100) and centrifugation. Additional lysis buffer was added to a total protein concentration of 0.3 mg/mL, and overexpression of Flag™-FKBP12 was verified by Western blot (data not shown). Small-molecule microarrays were serially incubated with clarified lysates, an anti-Flag™ M5 murine monoclonal primary antibody, and an Alexa Fluor® 647 goat-anti-mouse polyclonal secondary antibody. Antibodies were diluted to 0.5 μg/mL in PBST supplemented with 1.0% bovine serum albumin All incubations were performed for one hour at 4° C. Slides were briefly washed with PBST following incubations. After a brief rinse in distilled water, slides were dried by centrifugation, scanned, and analyzed as described above.

Protocol for Screening SMMs with Cellular Lysates

Transfection of HEK 293T Cells

1. Grow cells in DMEM/10% FBS+P/S+Glut until 70-90% confluent

2. Plate 5×10⁵ cells per well of a 6-well plate 24 (1 well=1 SMM)

3. Incubate 24 hours at 37° C.

4. Replace media with 2 mL warm DMEM/10% FBS

5. Prepare lipid transfection reaction in the following order:

| | |
|---|---|
| a. OptiMEM (Invitrogen) | 100 mL |
| b. Fugene 6 (Roche Diagnostics) | 3 uL |
| c. Plasmid DNA | 2 ug |

6. Tap to mix and incubate 15 minutes at room temperature

7. Gently pipette 100 uL transfection reaction dropwise around well

8. Incubate 36-48 hours at 37° C.

9. Monitor and record transfection efficiency by EGFP

Harvest of Transfected Cells

10. Harvest cells when EGFP efficiency >70% and intense

11. Aspirate medium and discard

12. Pipette 500 uL chilled PBS to each well

13. Gently liberate cell layer by repeated pipetting of PBS

14. Pool common cells in 15 mL Falcon tubes

15. Spin at 1,000 g and 4° C.×3 minutes

16. Discard supernatant

17. Gently resuspend in PBS

18. Repeat wash steps 15-17 three times total

19. Aliquot washed cell suspension in Eppendorf tubes

20. Spin at 1,000 g and 4° C.×3 minutes

21. Carefully discard supernatant maximally

22. Snap freeze cell pellets in EtOH/Dry Ice

23. Store at −80° C. until use

Preparation of Cellular Lysates

24. Thaw cell pellets on wet ice

25. Prepare MIPP Lysis Buffer with protease inhibitors and DTT (fresh)

26. Resuspend pellet in 100-200 uL Lysis Buffer

27. Incubate on ice for 15 minutes

28. Spin at 14,000 g and 4° C.×10 minutes

29. Decant cleared supernatant to new, chilled Eppendorf tube

30. Perform Bradford assay to assess protein concentration

31. Adjust with Lysis Buffer to achieve 0.3-1 ug/uL

Screening Printed Small Molecule Microarrays

32. Apply cellular lysate to slide surface (volume determined by method):

| | |
|---|---|
| a. Hybe Chamber | 1.4 mL |
| b. Parafilm | 0.3 mL |
| c. Coverslip | 0.15 mL |

33. Incubate at 4° C.×1 hour

34. Wash with chilled PBST—3×1 minute at 4° C.

35. Apply primary antibody (1:1000 dilution in PBST+1% BSA)

36. Incubate at 4° C.×1 hour

37. Wash with chilled PBST—3×1 minute at 4° C.

38. Apply secondary Cy5-labelled antibody (1:1000 dilution in PBST+1% BSA)

39. Incubate at 4° C.×1 hour

40. Wash with chilled PBST—3×3 minutes at 4° C.

41. Briefly rinse with ddH₂O

42. Centrifuge slides dry at 1000 rpm×1 minute

43. Scan at 635 with PMT voltage (500) and 100% Power

| MIPP Lysis Buffer | | 1.00 L | |
|---|---|---|---|
| NaH₂PO₄•2H₂0 | 20 mM | 3.12 | g |
| Na₃VO₄ | 1 mM | 0.184 | g |
| NaF | 5 mM | 0.08 | g (80 mg) |
| B-glycerophosphate | 25 mM | 5.48 | g |
| EGTA | 2 mM | 0.78 | g |
| EDTA | 2 mM | 0.72 | g |
| Triton X-100 | 0.5% | 2.5 | mL |

| | | |
|---|---|---|
| DTT | 1 mM | Fresh |
| pH | 7.2 | |

PBST = PBS + 0.1% Tween 20

Statistical Methods

Ten microarrays (5 treatment and 5 control) were used to determine interactions of printed small molecules with FKBP12-containing cell lysates. Each of the microarrays contained a total of 10,800 printed features. Of the 10,800 features on each microarray, 158 features contained only solvent and were used as negative controls to establish a threshold for intensity. The maximum fluorescence intensity value (i.e. threshold) over all the solvent cells (158×10=1,580) was found to be 2.243. Using this threshold value to dichotomize the data, a Fisher's Exact test was used to evaluate the hypothesis that the treatment cells had greater intensities than those of the control features. Contingency tables and p-values were generated for 104 solvent-only features in which at least one cell demonstrated fluorescence intensity above the threshold. Calculations were performed using the exact option in SAS (Cary, N.C.).

### Assessment of Binding of 1276-M08 by Surface Plasmon Resonance

SPR measurements were carried out on a Biacore S51 instrument (Biacore, Inc. Piscataway, N.J.). Each flow cell on a Biacore CM5 research grade sensor chips contains three addressable spots: two samples spots and a reference spot. Anti-GST was immobilized on spots 1 and 2 at a level of 13,000 Response Units (RU). Anti-GST diluted in 10 mM sodium acetate buffer (pH 5.0) was immobilized using the EDC/NHS attachment chemistry application wizard. The immobilization chemistry was quenched with ethanolamine. GST-tagged FKBP12 was captured on spot 1 at a level of 1,600 RU and recombinant GST was captured on spot 2.

Kinetic experiments were carried out in running buffer (24 mM Tris-HCl buffer, pH 7.4, 137 mM NaCl, 3 mM KCl, 0.005% (v/v) P20 surfactant and 5% (v/v) DMSO) at a flow rate of 30 μl/min Compounds were tested in duplicate at six different concentrations in a 1:2 dilution starting at 2.5 μM. Kinetic and equilibrium constants were calculated using Scrubber and ClampXP software (Center for Biomolecular Interaction, University of Utah). Binding data were double reference subtracted and globally fit using a 1:1 Langmuir binding model with the maximum number of binding sites determined experimentally with a synthetic ligand to FKBP12. Sensorgrams were normalized so that the maximum response would correspond to 100 RU on the y-axis.

Synthesis of FKBP12 Ligands 3a-3r

The carboxylic acid functionalized FKBP12-ligand 3f was synthesized according to the protocol in the following publication: Terence Keenan, David R. Yaeger, Nancy L. Courage, Carl T. Rollins, Mary Ellen Pavone, Victor M. Rivera, Wu Yang, Tao Guoy, Jane F. Amara, Tim Clackson, Michael Gilman and Dennis A. Holt; Bioorganic & Medicinal Chemistry 6 (1998) 1309-1335. 3f served as common intermediate for the other reported synthetic FKBP12-ligands (3a-e and 3g-q).

General Method A (Amide Coupling):

292 mg (0.5 mmol) carboxylic acid 3f was and 4 equivalents (20 equivalents in case of unprotected diamine) of the corresponding amine were dissolved in 5 mL methylene chloride. The reaction mixture was cooled to OC and 286 mg (0.55 mmol, 1.1 eq) PyBop were added in 1 mL methylene chloride. The reaction mixture was stirred at this temperature until all carboxylic acid was consumed (usually within 1 h). For workup, methylene chloride was added and the organic layer was washed with semi saturated sodium bicarbonate solution and water. The organic layer was dried over sodium sulfate and after filtration the solvent was removed under reduced pressure. The crude product was purified by preparative HPLC.

General Method B (Ester Formation):

146 mg (0.25 mmol) carboxylic acid 3f and 40 mg (0.3 mmol, 1.2 eq) N,N Dimethylaminopyridine were dissolved in 3 mL methylene chloride. The reaction mixture was cooled to OC and 62 mg (0.3 mmol) Dicyclohexylcarbodiimide were slowly added in 1 mL methylene chloride followed by 10 equivalents (2.5 mmol) of the corresponding diol. The reaction mixture was then warmed to room temperature and stirred for one hour. The precipitate was filtered of and washed with methylene chloride. The organic layers were combined and the solvent was removed under reduced pressure. The crude product was first purified on silica on an ISCO CombiFlash system (hexanes-ethyl acetate, gradient 10% to 100% ethyl acetate, detection 278 nm) followed by preparative HPLC.

Primary alcohol 3a: Method A
Secondary alcohol 3b: Method A
Tertiary alcohol 3c: Method B
Phenol 3d: Method A
Methyl ether 3e: Method A
Hydroxamic acid 3g: Method A
Alkyl 3h: Method A
Thiol 3i: Method A
Primary amine 3j: Method A
Secondary amine 3k: Method A
Indole 3l: Method A
Aniline 3m: Method A
3-PEG primary amine 3n: Method A
2-PEG primary alcohol 3o: Method B
3-PEG primary alcohol 3p: Method B
5-PEG primary alcohol 3q: Method B

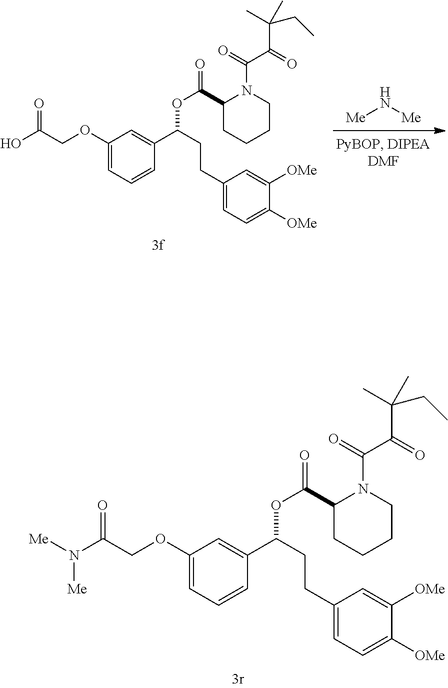

N,N-dimethyl amide AP1497 derivative 3r: A 10-mL round bottom flask was charged with 3f (10 mg, 17.2 μmol), and dried under high vacuum before addition of coupling reagents. Under an Ar atmosphere, the coupling reagents (1.4 equiv. N,N-dimethylamine, 1.6 equiv. PyBOP, 2.8 equiv. DIPEA in 3 mL anhydrous DMF) were added to the flask. The mixture was stirred under argon at ambient temperature for 14 hours and the reaction outcome was monitored by TLC. Upon completion, the reaction mixture was diluted with ethyl acetate (10 mL). The organic layer was washed with 2% KHSO₄ (aq), ddH₂O, brine and dried under anhydrous Na₂SO₄. The filtrate was concentrated under reduced pressure, and flash column chromatography (CHCl₃:MeOH=20:1) provided the desired product as a clear oil (11 mg, 92.3% isolated yield). ¹H NMR (500 MHz, CDCl₃) 8 ppm 7.27 (t, J=7.5 Hz, 1H), 6.96 (d, J=8 Hz, 1H), 6.95 (s, 1H), 6.89 (m, 1H), 6.78 (d, J=9 Hz, 1H), 6.68 (m, 2H), 5.78 (t, J=6 Hz), 5.31 (d, J=6 Hz, 1H), 4.70 (m, 2H), 3.86 (s, 3H), 3.85 (s, 3H), 3.65 (d, J=9.5 Hz, 1H), 3.16 (td, J=12.5, 2.5 Hz, 1H), 3.09 (s, 3H), 2.98 (s, 3H), 2.62-2.51 (m, 2H), 2.37 (d, J=12.5 Hz, 1H), 2.25 (m, 1H), 2.05 (m, 1H), 1.79-1.61 (m, 6H), 1.50 (qt, J=13, 4 Hz, 1H), 1.35 (qt, J=13, 4 Hz, 1H), 1.25-1.19 (m, 6H), 1.11 (d, J=6 Hz, 1H), 0.88 (t, J=8 Hz, 3H); ¹³C NMR (125 MHz, CDCl₃) 8 ppm 193.2, 186.3, 178.9, 170.0, 158.2, 141.4, 133.4, 130.0, 125.0, 120.1, 119.8, 114.3, 113.1, 111.7, 111.2, 67.4, 55.8, 51.3, 46.7, 44.1, 38.0, 35.7, 32.5, 31.2, 26.4, 24.9, 23.5, 23.1, 21.6, 21.2, 8.7; HRMS (TOF-ES+) calc. for C₃₄H₄₇N₂O₈Na (M+H)⁺, 611.3332 (1.6 ppm error).

### LCMS Data:

Carboxylic acid AP1497 derivative 3f, parent material for coupling reactions:

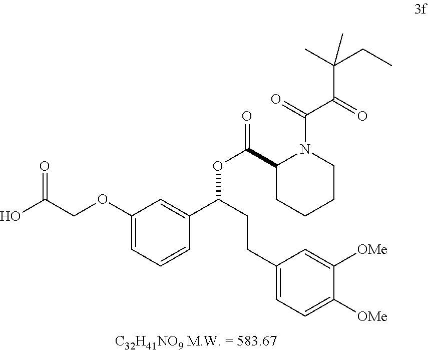

Primary Alcohol AP1497 Derivative 3a:

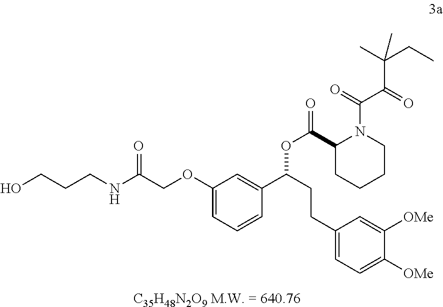

Secondary Alcohol AP1497 Derivative 3b:

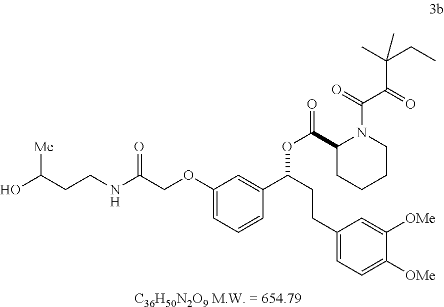

Tertiary Alcohol AP1497 Derivative 3c:

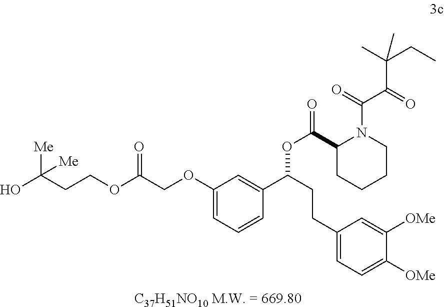

Phenol AP1497 Derivative 3d:

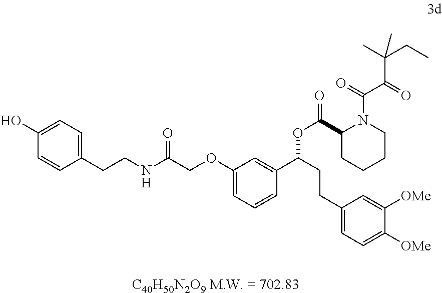

Methyl Ether AP1497 Derivative 3e:

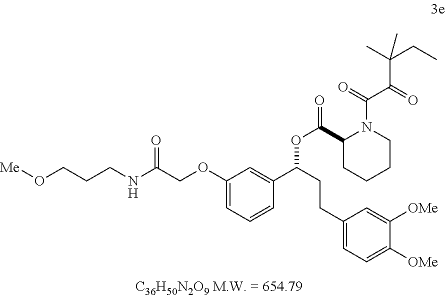

Hydroxamic Acid AP1497 Derivative 3g:

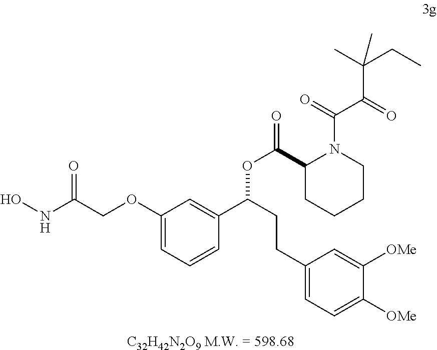

Propyl Amide AP1497 Derivative 3h:

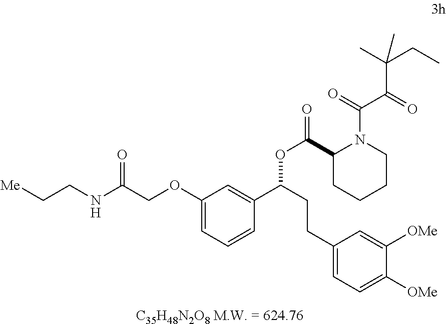

Thiol AP1497 Derivative 3i:

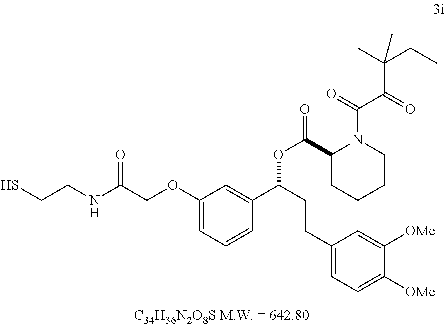

Primary Amine AP1497 Derivative 3j:

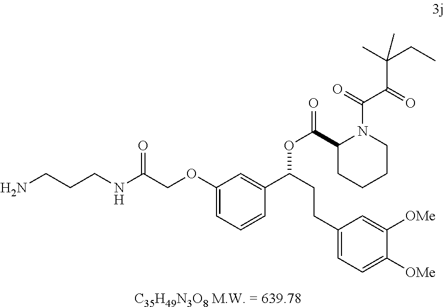

Secondary Amine AP1497 Derivative 3k:

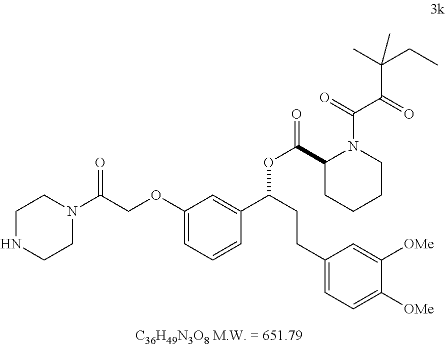

Indole AP1497 Derivative 3l:

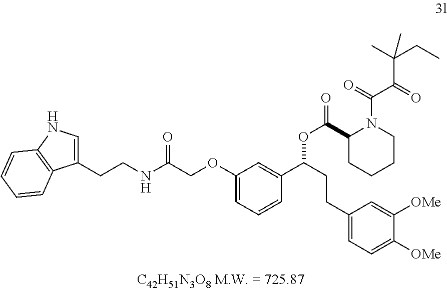

Aryl Amine AP1497 Derivative 3m:

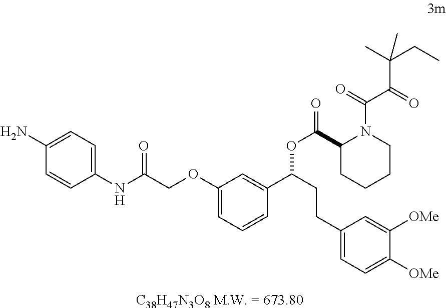

PEG Primary Amine AP1497 Derivative 3n:

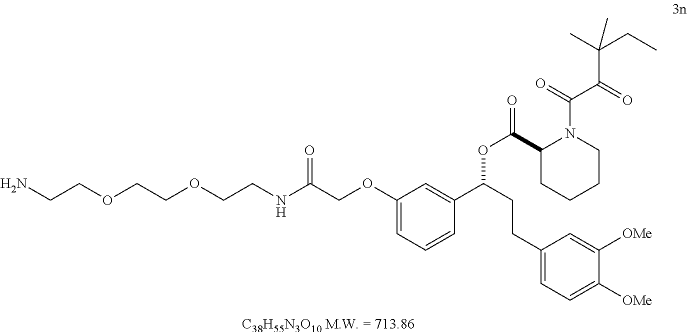

PEG Primary Alcohol (2O) AP1497 Derivative 3o:

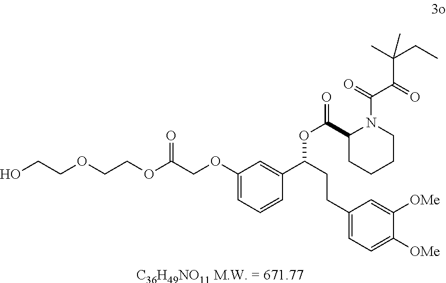

PEG Primary Alcohol (3O) AP1497 derivative 3p:

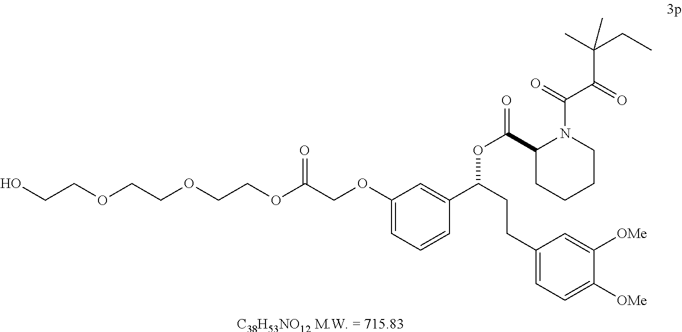

PEG Primary Alcohol (5O) AP1497 derivative 3q:

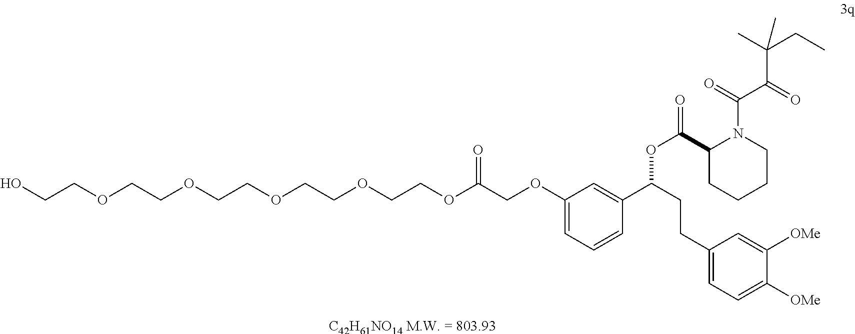

### Example 2

### A Method for the Covalent Capture and Screening of Diverse Small Molecules in a Microarray Format

This example describes a robust method for the covalent capture of small molecules with diverse reactive functional groups in microarray format and outlines a procedure for probing small molecule microarrays with proteins of interest. A vapor-catalyzed, isocyanate-mediated surface immobilization scheme is used to attach bioactive small molecules, natural products, and small molecules derived from diversity-oriented synthesis pathways. Additionally, an optimized methodology for screening small molecule microarrays with purified proteins and cellular lysates is described. Finally, a suggested model for data analysis that is compatible with commercially available software is provided. These procedures enable a platform capability for discovering novel interactions with potential application to immunoglobulin profiling, comparative analysis of cellular states and ligand discovery.

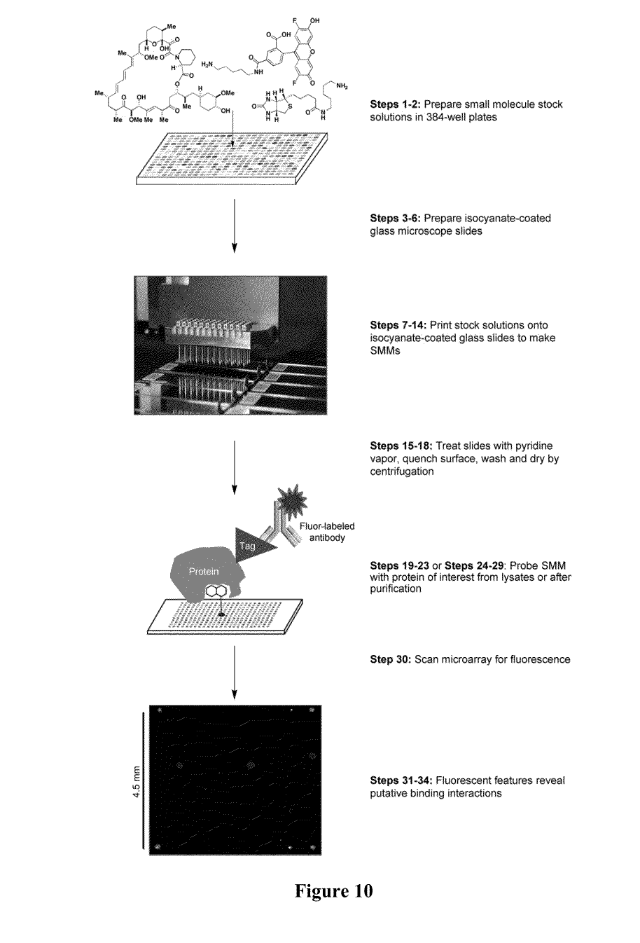

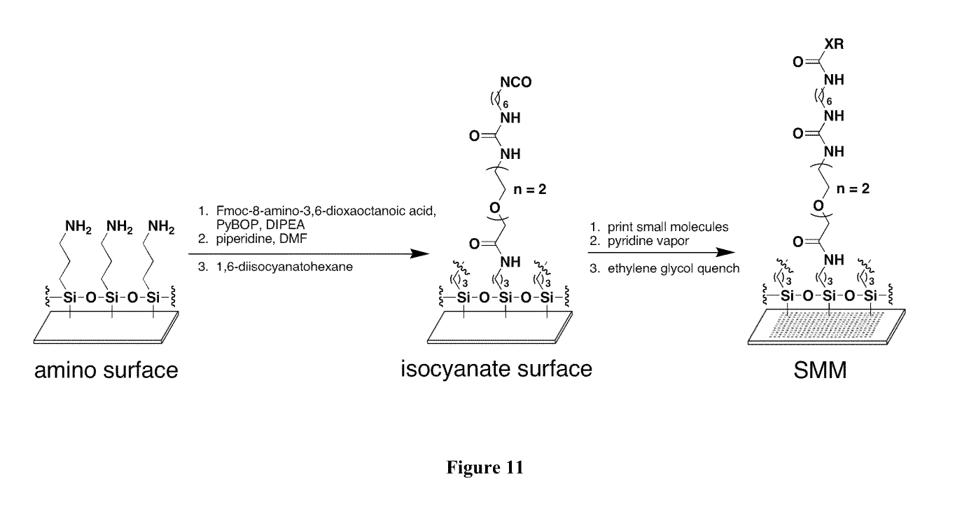

Here, we present a detailed, step-by-step description of this method for the covalent capture of diverse collections of small molecules using the vapor-catalyzed, isocyanate-mediated technique. A schematic diagram of this approach is provided in FIG. 10. Stock solutions of small molecules are arrayed in 384-well plate format. A protected polyethylene glycol (PEG) surface is prepared on glass microscope slides (FIG. 11). Following deprotection, 1,6-diisocyanatohexane is coupled to establish the reactive isocyanate surface. Small molecules are robotically printed and covalent attachment to the surface is then catalyzed by pyridine vapor. Quenched and washed slides are then stored dry for use in further experiments. The compatibility with complex natural products, products of diversity-oriented synthesis and bioactive small molecules, such as pharmaceutical agents, promises greatly to improve the quantity and structural diversity of printed small-molecule features.

This surface is experimentally compatible with assays involving clarified cellular lysates, frequently obviating the need for biochemical purification of a target. An optimized protocol for screening small-molecule microarrays with purified proteins and cellular lysates is also described. Following incubation with a small volume of the protein or lysate, slides are washed and then serially incubated with a primary antibody and labeled secondary antibody. Detection of binding interactions is determined quantitatively from data collected in triplicate using standard, commercially available software developed for the analysis of printed oligonucleotide arrays. Although not described here, candidate protein-ligand interactions discovered using this protocol are typically characterized using secondary binding assays involving fluorescence-based thermal shifts and surface plasmon resonance.

There are limitations to the methods of printing and detection described in this manuscript. First, many academic environments may have limited access to chemical libraries for screening. The investment of resources and training required to establish a functional robotic microarray printing platform may also pose institutional challenges. After an initial investment of $150,000 for equipment, the estimated cost of printing and screening SMMs is less than $20 per array. With respect to SMM screening, many research environments have access to all reagents and equipment necessary through microarray facilities aimed at the study of genomics.

The protocol described herein involves the use of several organic solvents and materials that require the use of appropriate safety equipment, such as safety glasses or gloves, and a properly ventilated fume hood. Notes from material safety data sheets (MSDS) are provided for selected reagents. All reactions and washes are performed in a fume hood. For more guidance on proper organic laboratory techniques please consult reference 17. Equipment and software are provided as examples. Alternative equipment and software may be used. The microarrays may be prepared in a microarray facility that is equipped with a properly enclosed and ventilated microarrayer as well as a neighboring fume hood. The small-molecule microarrays may be screened and scanned at any standard microarray facility. In Table 1, we have suggested printing several commercially available dyes and small molecules, including immunosuppressant natural products and known ligands to the protein FKBP12, as test cases. Applying the present protocol to these ligand-protein pairs will be of help in getting a handle on the procedure described herein.

The SMM printing and screening methodologies described herein provide a blueprint for the construction of a portable, robust, parallel platform for the discovery of novel protein-ligand interactions. Prior discoveries of small molecules targeting yeast transcription factors suggest that future applications to gene regulatory elements mediating disease phenotypes, such as neoplastic transformation, will enable the identification of tool compounds and leads for further pharmaceutical development. Compatibility of the slide surface with cellular lysates creates an additional opportunity to profile cellular states or complex mixtures such as serum immunoglobulins.

### Materials

### Reagents

Corning GAPS II coated glass slides (Fisher 07-200-006)
Fmoc-8-amino-3,6-dioxaoctanoic acid (NeoMPS, FA03202). Polyethylene glycol spacers of varying lengths (n=2-10 ethylene glycol units) have been successfully used with this protocol. Spacers of longer length (n>30) provide lower fluorescence intensity values and inconsistent spot morphologies.
(Benzotriazol-1-yloxy)tripyrrolidinophosphonium hexafluorophosphate, PyBOP® (Novabiochem, 01-62-0016)
Piperidine, redistilled (Sigma-Aldrich, 411027)
1,6-diisocyanatohexane (Aldrich, D124702)
Pyridine (Aldrich, 270970)
Ethylene glycol (Acros Organics, 295530010)
N,N-Dimethylformamide, DMF (Fisher Chemical, D131-4)
N,N-Diisopropylethylamine, DIPEA (Sigma-Aldrich, 550043)
Dimethyl sulfoxide (Acros Organics, 414880010)
Acetonitrile (Fisher Chemical, A998-4)
Tetrahydrofuran, THF, stabilized (Acros Organics, 164240025)
Texas Red® cadaverine (Invitrogen, T-2425)
Oregon Green® 488 cadaverine (Invitrogen, 0-10465)
Alexa Fluor® 647 cadaverine (Invitrogen, A-30679)
Rapamycin (LC Laboratories, R-5000)
FK506 (LC Laboratories, F-4900)
AP1497 was prepared as described in reference 18
FKBP12-6×His was prepared as described in reference 8
Alexa Fluor® 647 conjugate anti-SxHis antibody (Qiagen, 35370)
Cy5 mono-Reactive Antibody Labeling Dye Pack (GE Healthcare, PA25001)
293T Cells (ATCC, CRL-11268)
Lipofectamine 2000 transfection reagent (Invitrogen, 11668-019)
OptiMEM reduced serum, component-free medium (Invitrogen, 11058)
Flag-FKBP12 mammalian overexpression construct as described in reference 15
Anti-FLAG® M5 monoclonal mouse antibody (Sigma, F4042)
Anti-mouse goat secondary antibody, Alexa Fluor® 647 conjugate (Invitrogen, A-21237)
Anti-rabbit goat secondary antibody, Alexa Fluor® 647 conjugate (Invitrogen, A-21246)
Biotin (Sigma, B4501)
Biotin-PEG amine (Sigma, B9931)
Biotin cadaverine (Invitrogen, A-1594)
Streptavidin, Alexa Fluor® 647 conjugate (Invitrogen, S-32357)
Digoxin (Sigma, D6003)
Anti-digoxin mouse monoclonal antibody, clone DI-22, ascites fluid (Sigma, D8156)
Corticosterone (Fluka, 27840)
Anti-corticosterone rabbit antibody, whole antiserum (Sigma, C8784)
Protease inhibitor cocktail tablets (Roche, 11836170001)
Tris-buffered saline (TBS, 0.025 M Tris-HCl, 0.137 M NaCl, 0.003 M KCl, pH 7.4)
Tris-buffered saline with Tween-20 (TBST, 0.025 M Tris-HCl, 0.137 M NaCl, 0.003 M KCl, pH 7.4, 0.01% (v/v) Tween-20)
Phosphate-buffered saline (PBS, 0.012 M NaH₂PO₄, 0.137 M NaCl, 0.003 M KCl, pH 7.4)
Phosphate-buffered saline with Tween-20 (PBST, 0.012 M NaH₂PO₄, 0.137 M NaCl, 0.003 M KCl, pH 7.4, 0.01% (v/v) Tween-20)
MIPP lysis and incubation buffer (0.02 M NaH₂PO₄, 0.001 M Na₃VO₄, 0.005 M NaF, 0.025 M β-glycerophosphate, 0.002 M EGTA, 0.001 M DTT, 0.5% (v/v) Triton X-100, pH 7.2). Use of RIPA lysis and extraction buffer (0.025 M Tris-HCl, 0.15 M NaCl, 1% (v/v) NP-40, 1% (v/v) sodium deoxycholate, 0.1% (v/v) SDS, pH 7.6) results in the formation of an autofluorescent film on the slide surface that significantly decreases the signal-to-noise in the assay and should be avoided.

### Equipment

OmniGrid 100 Microarrayer (Genomic Solutions)
946 Printhead (Telechem International, 946PH48)
946 Micro spotting pins (Telechem International, 946 MP3)
384-well polypropylene natural microarray plates, cyclindrical wells (Abgene, AB-1055)
Thermal peelable plate seals (Velocityll, 06643001)
Desiccator dry storage box, acrylic (VWR, 24987-053)
Table-top centrifuge with microplate carriers
Low-particle nitrile gloves (VWR, 40101-222) (Use of some powdered gloves can result in autofluorescent residue on the microarrays.)
Bibulous paper (Fisher Scientific, 11-998)
Stainless steel 50-slide racks (Wheaton Scientific, 900404)
Large glass trough with stainless steel lid, 500 mL (Raymond A Lamb, E102-6)
Three-way glass vacuum valve with o-ring tip (Aldrich, Z271330)
Tygon R-3603 vacuum tubing
Glass vacuum desiccator (Aldrich, Z114340)
4-well rectangular polystyrene dishes (Nunc, 267061)
Square petri dishes, 100×100×15 mm (Nunc, 4021)
Parafilm® M (Fisher Scientific, 13-374-10)
Hybrislip™ hybridization covers, 60×22 mm (Invitrogen, H-18202)
Eppendorf tubes
Orbital platform shaker (VWR, 82004-958)
2-slide centrifuge for microarray drying (Sunergia Medical, MSC-T)
Genepix 4200A 4-laser slide scanner (Molecular Devices)
Genepix Pro 6.0 software (Molecular Devices)

### Reagent Setup

Small Molecules: Several small molecules that contain isocyanate-reactive functional groups are suggested as test molecules to evaluate the method (Table 1).

TABLE 1
Fluors and suggested protein-small molecule pairs for testing the protocol.

| Small | | Screening | | |
|---|---|---|---|---|
| Molecule | Protein | Concentration | K_{D} | Detection |
| Cy5 | — | — | — | Fluorescent dye |
| Oregon | — | — | — | Fluorescent dye |
| Green | | | | |
| Texas Red | — | — | — | Fluorescent dye |
| Biotin | Streptavidin-Alexa | 0.5 μg mL⁻¹ | 10⁻¹⁵M | Fluor-labeled protein |
| Derivatives | Fluor ® 647 | | | |
| AP1497 | FKBP12-6xHis | 1 μg mL⁻¹ | 18 nM | 1:1000 Fluor-labeled |
| | | | | anti-5xHis antibody |
| FK506 | FKBP12-6xHis | 1 μg mL⁻¹ | 3 nM | 1:1000 Fluor-labeled |
| | | | | anti-5xHis antibody |
| Rapamycin | FKBP12-6xHis | 1 μg mL⁻¹ | 0.5 nM | 1:1000 Fluor-labeled |
| | | | | anti-5xHis antibody |
| Corticosterone | Anti-corticosterone | 1:500 | nd | 1:1000 Fluor-labeled |
| | antibody | antiserum | | anti-rabbit secondary |
| | | | | antibody |
| Digoxin | Anti-digoxin antibody | 1:500 ascites | nd | 1:1000 Fluor-labeled |
| | | fluid | | anti-mouse secondary |
| | | | | antibody |

nd = not determined

Ordering information for the compounds is provided in the Reagents section. The small molecules should be diluted in DMSO to prepare 10 mM stock solutions for printing as described in Step 1.

Proteins: The suggested test molecules may be detected with a known protein or antibody partner (Table 1). Printed biotin derivatives may be detected using a commercially available streptavidin-fluor conjugate as described in Step 21 (Method A) and Step 22 (Method A). Corticosterone and digoxin may be detected using commercial antibodies against the compounds followed by labeled secondary antibodies as described in Step 21 (Method A) and Step 22 (Method B). AP1497, FK506, and rapamycin can be detected by incubation with epitope-tagged FKBP12 and a labeled antibody directed against the epitope tag as described in Step 21 (Method A) and Step 22 (Method B). Finally, a protocol for detecting this interaction using epitope-tagged FKBP12 from cell lysates, using a primary antibody and labeled secondary antibody, is described in Steps 24-29. Suggested screening concentrations and antibody dilutions for each test case are provided in Table 1. Standard buffers such as TBST or PBST may be used for all experiments.

### Equipment Setup

Customized Microarrayer Wash Station.

The standard OmniGrid 100 setup includes a sonicator for aqueous washing of the printing pins. For small-molecule microarrays, an organic solvent such as acetonitrile is used to wash away the compounds from the pins. The sonication station has been substituted with a stir plate and a recrystallizing dish containing acetonitrile. During each wash step, the printhead is dipped into the stirring acetonitrile dish for 5 seconds followed by 3 seconds at the vacuum drying station. For each pin dip, the wash dry cycle is repeated three times to minimize carryover of samples. Make sure that the stir bar does not create a deep vortex such that the pins do not make contact with solvent. Occasionally monitor the solvent level to ensure that the pins are effectively washed.

Typical Genepix Scanner Settings.

Pixel size: 10 μm; PMT voltages per laser: 635 nm ex. (red)=500-600, 594 nm ex. (yellow)=600, 532 nm ex. (green)=500-550, 488 nm ex. (blue)=400-500.

### Procedure

Preparation of Small-Molecule Stock Solutions for Printing
1. Dissolve small molecules of interest in DMSO. Typically, printing stock concentrations range from 1 mM to 10 mM. DMF is a suitable alternative solvent for preparing stock solutions. Stock solutions are stored at −20° C.
2. Transfer 5 μL of each stock solution to individual wells in a 384-well polypropylene microarray plate. For large sample numbers it is desirable to use a liquid transfer robot. Sealed stock plates are stored at −20° C. and undergo up to ten freeze-thaw cycles prior to liquid chromatography-mass spectrometry (LC-MS) analysis to monitor to the stability of compound stocks. The number of acceptable freeze-thaw cycles often depends on the nature of the small molecules that are printed. A typical set of printing stock plates is retired after twelve freeze-thaw cycles.

Preparation of Isocyanate-Coated Glass Microscope Slides
3. Place one hundred amino-functionalized GAPS II slides (Corning) into two stainless steel 50-slide racks. Submerge each rack in a large glass trough containing fresh PEG solution: Fmoc-8-amino-3,6-dioxaoctanoic acid (1 mM), PyBOP (2 mM), and DIPEA (0.5 mM) in 1 L of DMF. The solution should completely cover the slides. Incubate the slides in the PEG solution with stiffing at room temperature in a fume hood for at least 4 hours. The incubation is typically performed overnight.
4. Remove the racks from the PEG solution, and allow them to drip before briefly rinsing in DMF. Drip-dry the racks again, and place them into a clean tank containing 1% (v/v) piperidine in 1 L of DMF to remove the Fmoc group from the surface. The deprotection reaction is complete after 10 minutes at room temperature. The slides can be left in the deprotection solution overnight.
5. Remove the racks from the piperidine solution, drip dry, and wash for one minute in DMF with stirring. To install the isocyanate group on the surface of the slides, place the deprotected slides into troughs containing 1% (v/v) 1,6-diisocyanatohexane in DMF. Incubate the fully submerged slides in this solution with stirring for 30 minutes at room temperature.
6. Immerse the activated slides in DMF with stirring and wash for 3 minutes. Repeat with fresh DMF. Immerse the slides in THF with stiffing and wash for 2 minutes. This wash sequence effectively removes excess isocyanate reagent from the slides and will provide clean and dry slides. Slides are typically dried prior to printing so that excess solvents and reagents are not exposed to the microarrayer platform. Slides may be dried under a gentle stream of air for a minute or two after the final THF rinse. Otherwise, simply allow the THF to evaporate off for a few minutes.

Printing Small-Molecule Microarrays
7. Remove the compound stock plates from the freezer and allow them to thaw in a desiccator dry storage box.
8. Carefully place the dried and activated slides onto the microarrayer platform. Be sure that the slides are all in a common orientation with respect to the barcode sticker.
9. Load clean printing pins into the printhead being careful to avoid touching the tips of the pins.
10. Design the printing configuration using the OmniGrid 100 software. Printing from DMSO typically provides features with spot diameters around 150 μm. Using a center-to-center spacing of 300 μm comfortably allows 10,800 features to be printed in 15×15 subarrays using 48 pins.
11. Centrifuge all compound stock plates at 400 g for 1 minute using a Genevac HT-24 or standard benchtop centrifuge with microplate adapters. Plates should be centrifuged to be sure that all of the stock solution resides at the bottom of the well.
12. Insert a clean glass blot pad in one of the three microplate positions. Insert the first two compound stock plates into the remaining microplate holders. Be sure that all stock plates are placed on the microplate holders in the proper orientation with respect to well A01 to avoid inconsistencies between the actual printing sequence and the theoretical print sequence or GAL file.
13. Print compounds in desired array format. Instruct the arrayer to pre-spot 30 features at 400-μm center-to-center spacing on the blot pad for every sample pickup. Clean the blot pad with bibulous paper and methanol after printing every two plates. Printing solutions on a blot pad prior to spotting on the activated slides avoids excess solution from creating large spots on the first few slides of the print run.
14. After the print run is completed, leave the slides on the microarrayer platform for at least 10 minutes so that the printed samples will dry.
15. Move printed slides into the stainless steel slide racks. Place the racks in a vacuum desiccator attached to a 3-way glass valve through Tygon tubing in a ventilated chemical fume hood. The major outlet should be directed to the desiccator, through tubing, with one of the valves directed to a vacuum line, also through tubing. The other (closed) valve should be directed, through tubing, to a flask with 2 mL anhydrous pyridine. Evacuate the desiccator containing the slides. Keep the slides under vacuum for five minutes to assist the removal of any excess printing solvent. Close off the vacuum line and open the valve to the flask containing pyridine. The printed slides in the desiccator are then exposed to pyridine vapor for at least 2 hours. Pyridine catalyzes the covalent attachment of functional groups that are less reactive towards isocyanate. Finally, close off the pyridine line and evacuate the desiccator to dry the slides. The slides are typically exposed to pyridine vapor during an overnight incubation.
16. Remove the racks from the desiccator and immerse the dried slides in a solution of 5% (v/v) ethylene glycol and 0.1% (v/v) pyridine in DMF with stirring for 30 minutes to quench the isocyanate surface.
17. After the ethylene glycol quench, rinse the slides in DMF. Wash the slides in DMF for 1 hr with stiffing followed by two brief washes, 3 minutes each, in THF. Dry the slides by centrifugation.
18. Dried slides are packaged in 5-slide boxes sealed with parafilm. Microarrays can be stored for up to six months at −20° C. The arrays may be kept at 4° C. for several days.

Quality Control: Detecting Known Protein-Small Interactions
19. Pre-scan to see known fluorophores (listed in Table 1) and to identify autofluorescent compounds.
20. Prepare protein or antibody solution to be used to detect a known printed ligand (listed in Table 1) in TBST buffer that has been kept chilled at 4° C. Purified proteins and antibodies are typically screened in the range of 0.1 to 5.0 μg mL⁻¹. It is important to use a buffer that is appropriate for the protein of interest. Buffers should contain specific cofactors or reagents that are required for activity or stability. It is best to avoid autofluorescent additives. TBST and PBST are commonly used and provided as examples.
21. Incubate diluted protein with microarray at 4° C. for 1 hour. Two incubation methods are described below. Method A is used when protein is not in limited supply or if agitation is desirable (use the same protocol for antibody incubations that may follow). The inexpensive method B is used to minimize the amount of protein used in the binding assay. This method was used as an alternative to coverslips, which provide inconsistent results and areas of high background surrounding the edge of the coverslip:
   A) Dish Method
      (i) Place the microarray, printed face up, in the well of a 4-well rectangular dish.
      (ii) Gently pipet 3 mL protein solution onto the slide barcode sticker and let the solution spread out to cover the surface of the slide. Alternatively, three slides may be placed printed face up in a square Petri dish.
      (iii) Cover the dish with the lid and place on a rocking platform so that the solution is gently agitated over the surface of the slide. Alternatively, gently pipet 6 mL of protein solution into the dish and agitate.
   B) Parafilm Method
      (i) Cut a strip of parafilm and place on a smooth and flat surface such as a clean lab bench in a cold room or on a chilled flat surface for transfer into a laboratory refrigerator or cold room.
      (ii) Pipet 300 μL of protein solution onto the parafilm.
      (iii) Carefully place the microarray, printed face down, onto the drop so that the protein solution spreads out to cover the entire slide. Avoid introducing air bubbles in between the printed surface of the slide and the parafilm.
22. Carefully remove protein solution from the microarray. Briefly rinse excess protein solution from the slide using chilled TBST buffer (4° C.). For assays using a directly labeled fluorescent protein follow Method A. Follow Method B for assays involving detection through a labeled antibody.
   A) Direct Detection of Fluor-Labeled Proteins
   For a protein that is directly labeled with a fluorescent moiety (e.g., Alexa 647, fluorescein, GFP, etc.), wash each slide in 3 mL buffer for 2 minutes with agitation on a platform shaker or rocker. Repeat twice. Wash once with chilled TBS buffer (4° C.) for 1 minute and go to step 23.
   B) Antibody-Based Detection
   When using a fluor-labeled antibody-based detection (e.g., anti-His, anti-GST, anti-FLAG, etc), immediately apply the diluted antibody of interest in TBST or another suitable buffer and place the slide at 4° C. for 1 hour. Carefully remove fluor-labeled antibody solution from the microarray. Briefly rinse excess protein solution from the slide using chilled TBST buffer. Wash the slide in 3 mL buffer for 2 minutes with agitation. Repeat twice. Wash once with chilled TBS buffer for 2 minutes.
23. Dry slides by centrifugation using a slide centrifuge. The probed microarrays are ready for analysis. Ideally slides are scanned immediately after probing with protein. Dried slides may be stored at room temperature and in the dark for up to two days prior to scanning without significant deterioration in fluorescent signal.

Protein Binding Screens Using Cell Lysates
24. Transfect HEK-293T cells with a mammalian overexpression construct encoding an epitope-tagged protein of interest. Cells are seeded in a 6-well plate at 5×10⁵ cells per well, anticipating one well will be required per SMM incubation. A reliable, high level of expression has been achieved in this cell line with most commercially available lipid transfection reagents following provided technical protocols. Cells are typically harvested between 48-72 hours after transfection, at the time a well transfected with an EGFP vector achieves a stable, high degree of expression. Protein expression and detection may be validated by immunoblot. Where feasible, immunoprecipitated protein may be assessed for activity in an appropriate biochemical assay.
25. Harvest cells for storage or lysis. Adherent cells are washed twice in chilled PBS in 6-well plates, resuspended in 500 μL per well of chilled PBS and transferred to labeled Eppendorf tubes. Cells are pelleted by brief centrifugation and the supernatant is discarded. Pelleted cells are typically snap-frozen in liquid nitrogen and stored at −80° C. until use.
26. Prepare cellular lysates for incubation with small molecule arrays. Cell pellets are thawed on wet ice and resuspended promptly and gently in MIPP lysis buffer supplemented with protease inhibitors and fresh DTT (300 μL volume per source well). Incubate on ice for 15 minutes. Lysates are then clarified by centrifugation at 14,000 g for 10 minutes at 4° C. Immediately following centrifugation, decant supernatant to new, chilled Eppendorf tubes. Perform a protein quantification assay and adjust with lysis buffer to achieve 0.3 μg mL⁻¹. MIPP lysis buffer has been determined to minimize autofluorescence with arrays prepared as above. TGN and RIPA lysis buffers interfere with signal-to-noise in controlled experiments.
27. Incubate SMM with lysates using the methods described in Step 21 for one hour at 4° C. Wash with gentle rotation in chilled PBST for one minute, repeating three times.
28. Incubate SMM immediately with primary antibody for one hour at 4° C. For epitope-directed antibodies such as anti-FLAG or anti-His, a 1:1000 dilution in PBST supplemented with 0.1% BSA is suggested. Wash with gentle rotation in chilled PBST (4° C.) for three minutes, repeating three times.
29. Incubate SMM immediately with secondary antibody for one hour at 4° C. Dilutions of 1:1000 are appropriate for most commercial fluor-labeled antibody solutions. Wash with gentle rotation in chilled PBST for three minutes, repeating three times. Briefly rinse with distilled water and dry slides by centrifugation for one minute. The probed slides are ready for analysis.

Data Analysis
30. Scan slides using the Genepix 4200A slide scanner using the suggested settings.
31. Align the corresponding GAL file, translating microarray location to microplate location, to each scanned image using the Genepix Pro 6.0 software. Use the printed fluor markers to help align each subarray. Properly resize each GAL file feature to the diameter of the actual printed microarray feature and generate a Genepix results (GPR) file for each microarray.
32. Analyze results file to evaluate a) whether fluorescent dye markers are present, b) whether known ligands are present, c) whether marker compounds carryover to the next sample resulting in contamination of neighboring features, d) which compounds are autofluorescent at the experimental wavelengths, and e) whether there are new small molecules that bind to the protein or antibody applied to the microarray.
33. Assay positives are scored from triplicate experimental data based on deviation from the mock-treatment distribution defined by the features containing solvent only on each SMM. Fluorescence intensity is adjusted for background signal on a per-spot basis within the GenePix software, and this metric is used principally in the analysis.
34. Assay positives are then compared to triplicate experimental data collected from control experiments as appropriate. As this platform is capable of detecting interactions between small molecules and immunoglobulins, comparison to a buffer-only or control lysate experiment followed by antibody incubation is essential.

### Results

Each of the experimental steps outlined in this protocol have been optimized for performance, yield, and reproducibility so as to accommodate fabrication of arrays for screening by a number of interested investigators. Typically, an investigator can anticipate the successful immobilization of nearly 11,000 diverse compounds in microarray format on a glass microscope slide. En route to this outcome, we recommend “assay development” screens with fluorescent ligands as controls for the printing process and known high-affinity ligands to validate the platform in a screening context.

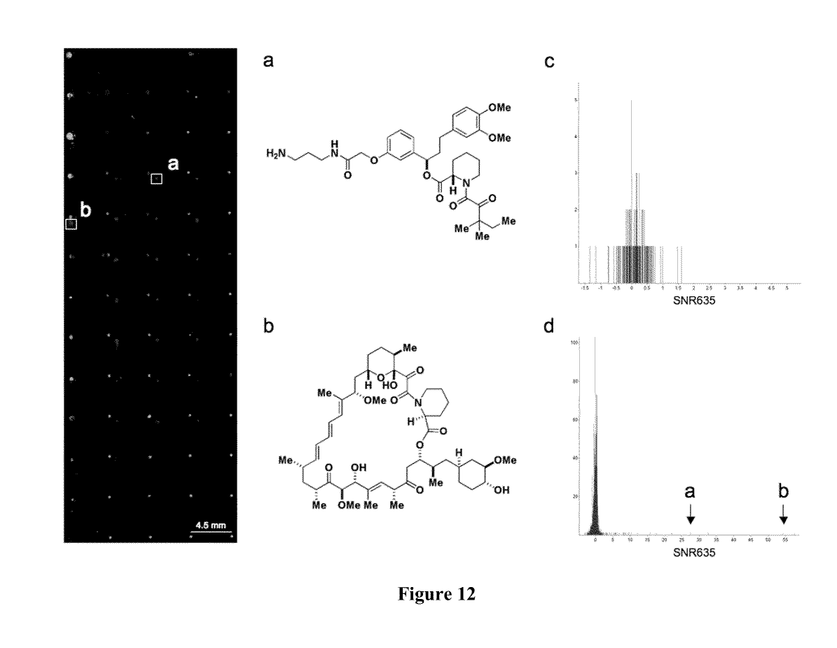

The optimized screening protocols for recombinant and transfected protein have proven reliable and robust as described. However with testing of new proteins, the influences of proper protein folding and stability in lysis buffer and the selection of epitope and antibody for detection are substantial. To illustrate the results anticipated from screening small molecule microarrays, we present data in FIG. 12 from a screen of a clarified cellular lysate from HEK-293T cells expressing Flag-FKBP12. In this experiment, a primary and secondary antibody detection scheme was used as described above. The array was scanned for fluorescence at 532 nm and 635 nm, false-colored green and red in this merged image, respectively. Assay positives appear in red. These data illustrate the anticipated detection of the small molecule ligand AP1497 (FIG. 12a), printed through a primary amine, and the natural product rapamycin (FIG. 12b), printed through a secondary alcohol. A histogram depicting the distribution of 635 nm fluorescence intensity corrected for local background of wells containing solvent alone is presented in FIG. 12c, illustrating the low noise of this experiment. A histogram depicting the same measurement from the printed small molecules on the array is presented in FIG. 12d. This figure illustrates the expected, comparable, low-intensity distribution of signal from inactive compounds and solvent. Additionally, as illustrated by the data highlighted with arrows, the AP1497 derivative and rapamycin appear as distinct assay positives by this analysis.

In sum, this protocol details an optimized strategy for printing diverse small molecules in microarray format and screening both purified proteins and complex mixtures. Using this platform, we have detected small molecule binders for protein targets with a range of affinities (2 nM to 50 μM), validated by surface plasmon resonance.

### Other Embodiments

Those of ordinary skill in the art will readily appreciate that the foregoing represents merely certain preferred embodiments of the invention. Various changes and modifications to the procedures and compositions described above can be made without departing from the spirit or scope of the present invention, as set forth in the following claims.

1. An array comprising:
a plurality of more than one type of chemical compound attached to a solid support through an isocyanate-derived linker or a isothiocyanate-derived linker, wherein the density of said array of compounds comprises at least 100 spots per cm².

2. The array of claim 1, wherein said array of chemical compounds comprises an array of small molecules.

3. The array of claim 1, wherein said array of chemical compounds comprises an array of non-oligomeric chemical compounds.

4. The array of claim 1, wherein said array of chemical compounds comprises an array of non-peptidic and non-oligomeric chemical compounds.

5. The array of claim 1, wherein said array of chemical compounds comprises an array of chemical compounds with a molecular weight less than 2000 g/mol.

6. The array of claim 1, wherein said array of chemical compounds comprises an array of chemical compounds, wherein the chemical compounds are not polynucleotides, peptides, or proteins.

7. The array of claim 1, wherein said array of chemical compounds comprises an array of chemical compounds, wherein the chemical compounds are biomolecules from a cell lysate.

8. The array of claim 1, wherein the chemical compounds include a functional group for attachment selected from the group consisting of primary alcohol, secondary alcohol, phenol, thiol, aniline, hydroxamic acid, primary amides, aliphatic amines, and sulfonamides.

9. The array of claim 1, wherein the chemical compounds include a functional group for attachement selected from the group consisting of primary alcohols, secondary alcohols, phenols, carboxylic acids, hydroxamic acids, thiols, and amines.

10. The array of claim 1, wherein said attachment is characterized in that the resulting linkage is robust enough so that the compounds are (1) not inadvertently cleaved during subsequent manipulation steps and (2) inert so that the functionalities employed do not interfere with subsequent manipulation steps.

11. The array of claim 1, wherein each of said chemical compounds in said array is attached to the solid support through a linkage generated by addition of a nucleophile to an isocyanate or isothiocyanate moiety.

12. The array of claim 11, wherein the addition is catalyzed by vapor.

13. The array of claim 11, wherein the addition is catalyzed by a nucleophile.

14. The array of claim 12, wherein the vapor is pyridine.

15. The array of claim 12, wherein the vapor is a volatile heterocyclic amine.

16. The array of claim 1, wherein the isocyanate-derived linker attaching compound to the support is of the formula: 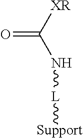
wherein
L is a substituted or unsubstituted, branched or unbranched, cyclic or acyclic aliphatic or heteroaliphatic linker;
X is N, S, or O; and
R is the chemical compound being attached to the solid support.

17. (canceled)

18. The array of claim 1, wherein the isocyanate-derived linker attaching compound to the support is of the formula: 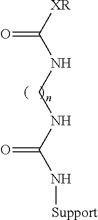
wherein
n is an integer between 1 and 12, inclusive;
X is O, S, or N; and
R is an attached compound.

19. The array of claim 1, wherein the isocyanate-derived linker attaching compound to the support is of the formula: 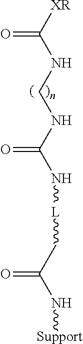
wherein
L is a substituted or unsubstituted, branched or unbranched, cyclic or acyclic aliphatic or heteroaliphatic linker;
n is an integer between 1 and 12, inclusive;
X is N, S, or O; and
R is the chemical compounds being attached to the solid support.

20. The array of claim 1, wherein the isocyanate-derived linker attaching compound to the support is of the formula: 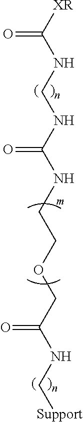
wherein
each occurrence of n is independently an integer between 1 and 20, inclusive;
m is an integer between 1 and 20, inclusive;
X is N, S, or O; and
R is the chemical compounds being attached to the solid support.

21. (canceled)

22. The array of claim 1, wherein the linker attaching compound to the solid support is shown below: 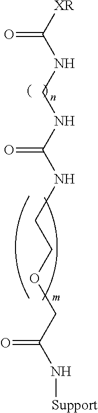
wherein n is an integer between 1 and 12, inclusive;
m is an integer between 1 and 200, inclusive;
X is O, S, or N; and
R is an attached compound.

23-51. (canceled)
